(19) United States
(12) Patent Application Publication
(10) Pub. No.: US 2024/0408054 A1
(43) Pub. Date: Dec. 12, 2024

(54) COMPOSITIONS AND METHODS FOR INCREASING EXPOSURE OF R(-)-MDA

(71) Applicant: ATAI Life Sciences AG, Berlin (DE)

(72) Inventors: Glenn SHORT, Scituate, MA (US); Srinivas G. RAO, Encinitas, CA (US)

(21) Appl. No.: 18/737,562

(22) Filed: Jun. 7, 2024

Related U.S. Application Data
(60) Provisional application No. 63/472,029, filed on Jun. 9, 2023.

(51) Int. Cl. A61K 31/36; A61K 9/00; A61K 31/137; A61K 31/166; A61K 31/341; A61K 31/343; A61K 31/381; A61K 31/4164; A61K 31/426; A61K 31/4525; A61K 31/454; A61K 31/506; A61K 31/5377; A61K 31/5513; A61K 31/58; A61K 31/635; A61P 25/22

(52) U.S. Cl. CPC A61K 31/36; A61K 9/0053; A61K 31/137; A61K 31/166; A61K 31/341; A61K 31/343; A61K 31/381; A61K 31/4164; A61K 31/426; A61K 31/4525; A61K 31/454; A61K 31/506; A61K 31/5377; A61K 31/5513; A61K 31/58; A61K 31/635; A61P 25/22

(57) ABSTRACT

Compositions and kits comprising enantiomerically enriched forms of MDMA, an inhibitor of CYP2D6, and a pharmaceutically acceptable carrier. Also disclosed are methods of increasing or prolonging exposure of R(−)-MDA in plasma or brain of a subject in need thereof.

### CROSS-REFERENCE TO RELATED APPLICATIONS

This application claims benefit from U.S. Provisional Application No. 63/472,029, filed Jun. 9, 2023, which is hereby incorporated by reference in its entirety for all purposes.

### BACKGROUND

3,4-Methylenedioxymethamphetamine (MDMA) is a psychoactive compound that affects mood, perception, and increases prosocial feelings and behaviors. It is a ring-substituted phenethylamine possessing a complex pharmacological profile that is dominated by its effects as a monoamine releaser and reuptake inhibitor. Its prominent serotonergic effects differentiate it from amphetamine and methamphetamine, which primarily act via dopaminergic and norepinephrine mechanisms of action. MDMA is considered the prototype for compounds referred to as entactogens, which means “to touch within” due to the induction of feelings of empathy and sociability. MDMA produces subjective effects that are unlike any of the classical psychostimulants or hallucinogens and is one of the few compounds capable of reliably producing prosocial behavioral states.

Racemic MDMA possesses a complex pharmacological profile as a result of enhanced triple monoamine (serotonin, norepinephrine, and dopamine) release coupled with inhibition of reuptake, blockade of vesicular storage of monoamines, inhibition of neurotransmitter oxidation by MAO-A, and a reversal of 5-HT transport into the neuron. These effects result in a net increase of monoamines in the synaptic cleft and prolong the duration of monoaminergic neurotransmission. MDMA also affects hormone secretion, promoting the release of oxytocin and arginine vasopressin (AVP).

MDMA has two stereoisomers S(+)-MDMA and R(−)-MDMA. The enantiomers have been shown to impact the monoaminergic targets of MDMA differently, resulting in pharmacological activity of S(+)-MDMA resembling those of psychostimulants, including increases in motor activity and euphoria, and R(−)-MDMA pharmacologic activity inducing effects closer to classical psychedelics, such as alteration in perception and ego-dissolution. The differing pharmacological and toxicological profile of the enantiomers indicate that R(−)-MDMA can provide an improved therapeutic index, offering the therapeutic effects of racemic MDMA but with a reduced side effect profile.

The metabolism of MDMA is complex and CYP-mediated metabolism in particular limits the overall exposure of R(−)-MDA and resulting psychedelic subjective effects that may promote rapid antidepressant effects and long term neuroplasticity that may be therapeutically useful.

The disclosure addresses the continuing need for therapeutics that are effective in modulating the exposure to R(−)-MDA.

### SUMMARY

The compositions, kits, and methods described herein comprise enantiomerically enriched forms of MDMA that exhibit desirable pharmacological profiles, such as providing increased or prolonged exposure of R(−)-MDA in the plasma or brain of a subject in need thereof.

In aspects, the disclosure provides a pharmaceutical composition comprising enantiomerically enriched R(−)-MDMA relative to S(+)-MDMA, or a pharmaceutically acceptable salt thereof, an inhibitor of CYP2D6, and a pharmaceutically acceptable carrier, wherein the enantiomeric excess of R(−)-MDMA is from about 50.01% to about 100% R(−)-MDMA. In aspects, the enantiomeric excess of R(−)-MDMA is from about 65% to about 100% R(−)-MDMA.

In aspects, the disclosure provides a kit comprising enantiomerically enriched MDMA comprising R(−)-MDMA in an enantiomeric excess relative to S(+)-MDMA, or a pharmaceutically acceptable salt thereof, and an inhibitor of CYP2D6, wherein the enantiomeric excess of R(−)-MDMA is from about 50.01% to about 100% R(−)-MDMA. In aspects, the enantiomeric excess of R(−)-MDMA is from about 65% to about 100% R(−)-MDMA.

In aspects, the ratio of the inhibitor of CYP2D6 to R(−)-MDMA in the composition is from about 1:1 to about 1:500. In other aspects, the ratio of the inhibitor of CYP2D6 to R(−)-MDMA in the composition is from about 1:1 to about 1:25.

In aspects, the inhibitor of CYP2D6 comprises a reversible CYP2D6 inhibitor. In aspects, the reversible inhibitor of CYP2D6 comprises quinidine, bupropion, fluoxetine, terbinafine, perhexiline, abiraterone, cinacalcet, duloxetine, lorcaserin, mirabegron, amiodarone, celecoxib, clobazam, cobicistat, desvenlafaxine, diltiazem, diphenhydramine, Echinacea, febuxostat, fluvoxamine, gefitinib, hydralazine, hydroxychloroquine, imatinib, labetalol, methadone, pazopanib, propafenone, ranitidine, ritonavir, sertraline, telithromycin, verapamil, vemurafenib, terfenadine, progesterone, testosterone, lansoprazole, olanzapine, chlorpromazine, fluphenazine, haloperidol, thioridazine, risperidone, clozapine, trifluperidol, Panax ginseng, or Ginko biloba. In embodiments, the reversible inhibitor of CYP2D6 can include quinidine, terbinafine, perhexiline, abiraterone, cinacalcet, duloxetine, lorcaserin, mirabegron, amiodarone, celecoxib, clobazam, cobicistat, desvenlafaxine, diltiazem, diphenhydramine, Echinacea, febuxostat, fluvoxamine, gefitinib, hydralazine, hydroxychloroquine, imatinib, labetalol, methadone, pazopanib, propafenone, ranitidine, ritonavir, telithromycin, verapamil, vemurafenib, terfenadine, progesterone, testosterone, lansoprazole, olanzapine, chlorpromazine, fluphenazine, haloperidol, thioridazine, risperidone, clozapine, trifluperidol, Panax ginseng, or Ginko biloba. In some alternative embodiments, the reversible inhibitor of CYP2D6 is not selected from bupropion, fluoxetine, and/or sertraline.

In embodiments, compositions in accordance with the disclosure comprise an inhibitor of CYP2D6 that is an irreversible CYP2D6 inhibitor. In embodiments, the irreversible CYP2D6inhibitor comprise cimetidine, pimozide, methamphetamine, metoclopramide, paroxetine, or desethylamiodarone. In embodiments, the irreversible CYP2D6 inhibitor comprise cimetidine, pimozide, methamphetamine, metoclopramide, or desethylamiodarone. In some further alternative embodiments, the irreversible CYP2D6 inhibitor is not paroxetine.

In aspects, the disclosure provides a kit comprising (i) a first composition comprising racemic MDMA or MDMA comprising S(+)-MDMA in an enantiomeric excess relative to R(−)-MDMA, or a pharmaceutically acceptable salt thereof, and a pharmaceutically acceptable carrier; and (ii) a second composition comprising enantiomerically enriched MDMA comprising R(−)-MDMA in an enantiomeric excess relative to S(+)-MDMA, or a pharmaceutically acceptable salt thereof, and a pharmaceutically acceptable carrier.

In aspects, the first composition comprises racemic MDMA. In other aspects, the first composition comprises S(+)-MDMA in an enantiomeric excess relative to R(−)-MDMA.

In aspects, the first composition comprises S(+)-MDMA in an enantiomeric excess relative to R(−)-MDMA in a total amount of about 50.01% to about 100% S(+)-MDMA. In other aspects, the first composition comprises S(+)-MDMA in an enantiomeric excess relative to R(−)-MDMA in a total amount of about 65% to about 100% S(+)-MDMA. In other aspects, the first composition comprises S(+)-MDMA in an enantiomerically pure form.

In aspects, the second composition comprises R(−)-MDMA in an enantiomeric excess relative to S(+)-MDMA in a total amount of about 50.01% to about 100% R(−)-MDMA. In other aspects, the second composition comprises R(−)-MDMA in an enantiomeric excess relative to S(+)-MDMA in a total amount of at least about 90% to about 100% R(−)-MDMA. In other aspects, the second composition comprises R(−)-MDMA in an enantiomerically pure form.

In aspects, the combined amount of S(+)-MDMA and R(−)-MDMA in the first composition is effective to partially inhibit CYP2D6 metabolism of MDMA. In embodiments, the weight ratio of the first composition to the second composition is from about 1:1 to about 1:1000. In embodiments, the weight ratio of the first composition to the second composition is from about 1:1 to about 1:25.

In aspects, the disclosure provides a method for sustaining responses to R(−)-MDA exposure in a subject comprising administering to the subject a therapeutically effective amount of a composition or kit in accordance with the disclosure.

In aspects, the subject has a CYP2D6 metabolic phenotype identified as ultra-rapid, extensive or efficient, or intermediate phenotype.

In aspects, the disclosure provides a method of increasing or prolonging exposure of R(−)-MDA in plasma or brain of a subject in need thereof, the method comprising administering to the subject: a therapeutically effective amount of a CYP2D6 inhibitor in an amount effective to inhibit CYP2D6 metabolism of MDMA, and a therapeutically effective amount of enantiomerically enriched R(−)-MDMA, or a pharmaceutically acceptable salt thereof.

In aspects, the disclosure provides a method of increasing or prolonging exposure of R(−)-MDA in plasma or brain of a subject in need thereof, the method comprising administering to the subject a first dose of racemic MDMA or MDMA comprising S(+)-MDMA in an enantiomeric excess relative to R(−)-MDMA, or a pharmaceutically acceptable salt thereof, in an amount effective to inhibit CYP2D6 metabolism of MDMA, and a second dose of enantiomerically enriched MDMA comprising R(−)-MDMA in an enantiomeric excess relative to S(+)-MDMA, or a pharmaceutically acceptable salt thereof.

In aspects, the R(−)-MDMA comprises at least 90% to about 100% of the total amount of MDMA.

In aspects, the CYP2D6 inhibitor and the enantiomerically enriched MDMA are administered concurrently, separately, or a combination thereof. In aspects, the CYP2D6 inhibitor is administered within about 12 hours of administration of the enantiomerically enriched MDMA.

In aspects, the ratio of the inhibitor of CYP2D6 to enantiomerically enriched MDMA is from about 1:1 to about 1:500. In other aspects, the ratio of the inhibitor of CYP2D6 to enantiomerically enriched MDMA is from about 1:1 to about 1:25.

In aspects, the disclosure provides a method for treating a social anxiety disorder in a subject comprising administering to the subject a therapeutically effective amount of the compositions or kits described herein. In embodiments, the CYP2D6 inhibitor is bupropion. In embodiments, the CYP2D6 inhibitor is quinidine. In embodiments, the effective amount of the inhibitor of CYP2D6 comprises a dose ranging from from about 0.1 mg to about 750 mg. In embodiments, the effective amount of the enantiomerically enriched MDMA comprises a dose ranging from 1 mg to about 750 mg MDMA, or an equivalent dose of the pharmaceutically acceptable salt thereof. In embodiments, the CYP2D6 inhibitor and the R(−)-MDMA are administered concurrently, separately, or a combination thereof.

In aspects, the disclosure provides a method for treating a social anxiety disorder in a subject comprising administering to the subject a therapeutically effective amount of the kits described herein. In embodiments, the effective amount of the first composition comprises a dose ranging from from about 0.1 mg to about 750 mg, or an equivalent dose of the pharmaceutically acceptable salt thereof. In embodiments, the effective amount of the second composition comprises a dose ranging from 1 mg to about 750 mg MDMA, or an equivalent dose of the pharmaceutically acceptable salt thereof. In embodiments, the first composition and the second composition are administered concurrently, separately, or a combination thereof.

Other aspects and embodiments will be apparent to one skilled in the art in light of the detailed description and Examples that follow.

### BRIEF DESCRIPTION OF THE DRAWINGS

FIGS. 1A-1B shows the SIRS (1A) as a function of R(−)-MDMA doses, with apparent maxima preceding pharmacokinetic Tmax serum response (1B).

FIG. 2 shows visual analog scale results for subjects at various dosages and placebo.

FIG. 3 shows the changes from baseline in happiness reaction time for subjects at various dosages and placebo.

FIG. 4 shows the changes from baseline for near 3.5 hours in MET LSmean scores for subjects at various dosages and placebo.

FIGS. 5A-5D show the changes from baseline in the concentration of mature brain-derived neurotrophic factor (FIG. 5A), prolactin (FIG. 5B), oxytocin (FIG. 5C), and norepinephrine (FIG. 5D).

FIGS. 6A-6B show responses (FIG. 6A) and statistical analysis (FIG. 6B) for 5D-ASC measurements for subjects at various dosages and placebo.

FIGS. 7A-7B show responses (FIG. 7A) and statistical analysis (FIG. 7B) for 11D-ASC measurements for subjects at various dosages and placebo.

FIG. 8 shows a plot of 11D-ASC measurements for R(−)-MDMA at indicated dosages and placebo.

FIG. 9 shows a plot of 11D-ASC measurements for R(−)-MDMA at 225 mg and 175 mg dosages, as well as psilocybin (at 20-30 mg), racemic MDMA (at 125 mg), and intravenous dimethyltryptamine (IV-DMT) at (0.667 mg/min.).

FIGS. 10A-10B show responses (FIG. 10A) and statistical analysis (FIG. 10B) for Emotional Breakthrough Inventory (EBI) observations with R(−)-MDMA for subjects at various dosages and placebo.

FIG. 11 shows changes from baseline 6.5 hours post dose in Challenging Experiences Questionnaire (CEQ) for subjects at various dosages and placebo.

### DETAILED DESCRIPTION

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example, in a list of numerical values such as “about 49, about 50, about 55, . . . ”, “about 50” means a range extending to less than half the interval(s) between the preceding and subsequent values, e.g., more than 49.5 to less than 50.5.

The terms “administer,” “administering” or “administration” as used herein refer to administering a compound or pharmaceutically acceptable salt of the compound or a composition or formulation comprising the compound or pharmaceutically acceptable salt of the compound to a patient.

The term “carrier” or “vehicle” as used interchangeably herein encompasses carriers, excipients, adjuvants, and diluents or a combination of any of the foregoing, meaning a material, composition or vehicle, such as a liquid or solid filler, diluent, excipient, solvent or encapsulating material involved in carrying or transporting a pharmaceutical agent from one organ, or portion of the body, to another organ or portion of the body. In addition to the adjuvants, excipients and diluents known to one skilled in the art, the carrier includes nanoparticles of organic and inorganic nature.

The terms “effective amount” and “therapeutically effective amount” are used interchangeably in this disclosure and refer to an amount of a compound, or a salt thereof, (or pharmaceutical composition containing the compound or salt) that, when administered to a patient, is capable of performing the intended result.

The term “MDMA,” without any further description, refers to racemic 3,4-methylenedioxymethamphetamine. MDMA has two stereoisomers, S(+)-MDMA and R(−)-MDMA. As described herein, enantiomerically enriched forms of MDMA or enantiomerically enriched MDMA comprise an unequal amount of the R(−)-MDMA and S(+)-MDMA enantiomers.

The phrase “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “salts” as used herein embraces pharmaceutically acceptable salts commonly used to form alkali metal salts of free acids and to form addition salts of free bases. Salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Base addition salts include but are not limited to, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e. g., lysine and arginine dicyclohexylamine and the like. Examples of metal salts include lithium, sodium, potassium, magnesium, calcium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Examples of organic bases include lysine, arginine, guanidine, diethanolamine, choline and the like. Those skilled in the art will further recognize that acid addition salts may be prepared by reaction of the compounds with the appropriate inorganic or organic acid via any of a number of known methods.

The term “therapeutic effect” as used herein refers to a desired or beneficial effect provided by the method and/or the composition.

The term “treating” as used herein with regard to a patient, refers to improving at least one symptom of the patient's disorder. Treating can be improving, or at least partially ameliorating a disorder or an associated symptom of a disorder.

The term “sustaining responses to R(−)-MDA” as used herein refers to extending pharmacodynamic effects of R(−)-MDA over one or more doses of the compositions or kits described herein to the subject. The pharmacodynmic effects of R(−)-MDA may be measured in any one of the methods provided herein or otherwise known in the art.

### Compositions and Kits

In aspects, the compositions of the present disclosure comprise MDMA (e.g., R(−)- and/or S(+)-MDMA, preferably R(−)-MDMA, in enantiomerically enriched or enantiomerically pure forms). In embodimetns, the compositions further comprise an inhibitor of CYP2D6 and a pharmaceutically acceptable carrier. Thus, in embodiments, compositions in accordance with the disclosure comprise R(−)-MDMA in an enantiomeric excess relative to S(+)-MDMA, or a pharmaceutically acceptable salt thereof, an inhibitor of CYP2D6, and a pharmaceutically acceptable carrier, wherein the amount of R(−)-MDMA in an enantiomeric excess relative to S(+)-MDMA is from about 50.01% to about 100% R(−)-MDMA. In embodiments, the amount of R(−)-MDMA in an enantiomeric excess relative to S(+)-MDMA may be from about 65% to about 100% R(−)-MDMA. In embodiments, 100% R(−)-MDMA comprise an enantiomerically pure form of R(−)-MDMA as measured by chiral HPLC.

Kits in accordance with the disclosure comprise (i) an inhibitor of CYP2D6; and (ii) enantiomerically enriched MDMA comprising R(−)-MDMA in an enantiomeric excess relative to S(+)-MDMA, or a pharmaceutically acceptable salt thereof. In embodiments, the amount of R(−)-MDMA in an enantiomeric excess relative to S(+)-MDMA is from about 50.01% to about 100% R(−)-MDMA. In embodiments, the amount of R(−)-MDMA in an enantiomeric excess relative to S(+)-MDMA may be from about 65% to about 100% R(−)-MDMA.

In embodiments, compositions and kits in accordance with the disclosure comprise R(−)-MDMA in an enantiomeric enrichment (or purity) of ≥90%, ≥95%, ≥96%, ≥97%, ≥98%, ≥ 99%, ≥99.5%, ≥99.9%, ≥99.99%, or 100% (i.e., relative to total MDMA).

In embodiments, wherein the R(−)-MDMA enantiomer is in excess compared to the S(+)-MDMA enantiomer, the composition comprises a weight ratio of R(−)-MDMA to S(+)-MDMA that is greater than 1. For example, the composition comprises a weight ratio of R(−)-MDMA to S(+)-MDMA ranging from about 50.01:49.99 to about 100:0 (e.g., about 50.1:49.9, about 50.5:49.5, about 51:49, about 52:48, about 53:47, about 54:46, about 55:45, about 56:44, about 57:43, about 58:42, about 59:41, about 60:40, about 61:39, about 62:38, about 63:37, about 64:36, about 65:35, about 66:34, about 67:33, about 68:32, about 69:31, about 70:30, about 71:29, about 72:28, about 73:27, about 74:26, about 75:25, about 76:24, about 77:23, about 78:22, about 79:21, about 80:20, about 81:19, about 82:18, about 83:17, about 84:16, about 85:15, about 86:14, about 87:13, about 88:12, about 89:11, about 90:10, about 91:9, about 92:8, about 93:7, about 94:6, about 95:5, about 96:4, about 97:3, about 98:2, about 99:1, about 99.5:0.5, about 99.9:0.1, or about 100:0, among other ratios within these enumerated ratios).

In embodiments, compositions and kits in accordance with the disclosure comprise a weight ratio of R(−)-MDMA to S(+)-MDMA ranging from about 90:10 to about 100:0 (e.g., a weight ratio of R(−)-MDMA to S(+)-MDMA of about 90:10, 91:9, about 92:8, about 93:7, about 94:6, about 95:5, about 96:4, about 97:3, about 98:2, about 99:1, about 99.5:0.5, about 99.9:0.1, or about 100:0, amount other ratios within these enumberated ratios). In embodiments, the composition comprises a weight ratio of R(−)-MDMA to S(+)-MDMA ranging from about 99:1 to about 100:0. For example, the weight ratio of R(−)-MDMA to S(+)-MDMA comprises about 99.1:0.9, about 99.2 to 0.8, about 99.3 to 0.7, about 99.4 to 0.6, about 99.5 to 0.5, about 99.6, about to 0.4, about 99.7 to 0.3, about 99.8 to 0.2, about 99.9 to 0.1, or 100:0.

As described above, the compositions and kits of the disclosure may further comprise an inhibitor of CYP2D6. In embodiments, the composition and kit comprises a weight ratio of the inhibitor of CYP2D6 to enantiomerically enriched MDMA that is about 1:1 or less than 1. For example, the composition and kit comprises a weight ratio of the inhibitor of CYP2D6 to enantiomerically enriched MDMA ranging from about 1:1 to about 1:500 (e.g., about 1:2, about 1:5, about 1:10, about 1:20, about 1:30, about 1:40, about 1:50, about 1:60, about 1:70, about 1:80, about 1:90, about 1:100, about 1:110, about 1:120, about 1:30, about 1:140, about 1:150, about 1:160, about 1:170, about 1:180, about 1:190, about 1:200, about 1:210, about 1:220, about 1:230, about 1:240, about 1:250, about 1:260, about 1:270, about 1:280, about 1:290, about 1:300, about 1:310, about 1:320, about 1:330, about 1:340, about 1:350, about 1:360, about 1:370, about 1:380, about 1:390, about 1:400, about 1:410, about 1:420, about 1:430, about 1:440, about 1:450, about 1:460, about 1:470, about 1:480, about 1:490, or about 1:500, among other ratios within these enumerated ratios).

In embodiments, compositions and kits in accordance with the disclosure comprise an inhibitor of CYP2D6, wherein the weight ratio of the inhibitor of CYP2D6 to enantiomerically enriched MDMA ranging from about 1:1 to about 1:25 (e.g., about 1:1, about 1:2, about 1:5, about 1:7, about 1:10, about 1:12, about 1:15, about 1:17, about 1:20, about 1:22, or about 1:25, among other ratios within these enumerated ratios).

In embodiments, compositions and kits in accordance with the disclosure comprise an inhibitor of CYP2D6 that is a reversible inhibitor. In aspects, the reversible CYP2D6 inhibitor will prevent further metabolism of R(−)-MDA. In embodiments, the reversible inhibitor of CYP2D6 comprise quinidine, bupropion, fluoxetine, terbinafine, perhexiline, abiraterone, cinacalcet, duloxetine, lorcaserin, mirabegron, amiodarone, celecoxib, clobazam, cobicistat, desvenlafaxine, diltiazem, diphenhydramine, Echinacea, febuxostat, fluvoxamine, gefitinib, hydralazine, hydroxychloroquine, imatinib, labetalol, methadone, pazopanib, propafenone, ranitidine, ritonavir, sertraline, telithromycin, verapamil, vemurafenib, terfenadine, progesterone, testosterone, lansoprazole, olanzapine, chlorpromazine, fluphenazine, haloperidol, thioridazine, risperidone, clozapine, trifluperidol, Panax ginseng, or Ginko biloba. In embodiments, the reversible inhibitor of CYP2D6 comprise quinidine, terbinafine, perhexiline, abiraterone, cinacalcet, duloxetine, lorcaserin, mirabegron, amiodarone, celecoxib, clobazam, cobicistat, desvenlafaxine, diltiazem, diphenhydramine, Echinacea, febuxostat, fluvoxamine, gefitinib, hydralazine, hydroxychloroquine, imatinib, labetalol, methadone, pazopanib, propafenone, ranitidine, ritonavir, telithromycin, verapamil, vemurafenib, terfenadine, progesterone, testosterone, lansoprazole, olanzapine, chlorpromazine, fluphenazine, haloperidol, thioridazine, risperidone, clozapine, trifluperidol, Panax ginseng, or Ginko biloba. In embodiments, the CYP2D6 inhibitor is bupropion. In embodiments, the CYP2D6 inhibitor is quinidine.

In some further alternative embodiments, compositions and kits in accordance with the disclosure comprise an inhibitor of CYP2D6 that is a reversible inhibitor, wherein the reversible CYP2D6 inhibitor is not fluoxetine, sertraline, escitalopram, and bupropion.

In embodiments, compositions and kits in accordance with the disclosure comprise an inhibitor of CYP2D6 that is an irreversible CYP2D6 inhibitor. In embodiments, the irreversible CYP2D6 inhibitor comprise cimetidine, pimozide, methamphetamine, metoclopramide, paroxetine, or desethylamiodarone. In embodiments, the irreversible CYP2D6 inhibitor comprise cimetidine, pimozide, methamphetamine, metoclopramide, or desethylamiodarone.

In embodiments, compositions and kits in accordance with the disclosure comprise an inhibitor of CYP2D6 that is an irreversible inhibitor, wherein the irreversible CYP2D6 inhibitor is not paroxetine.

In aspects, the disclosure provides a kit, or article of manufacture, that comprise (i) a first composition comprising racemic MDMA or MDMA comprising S(+)-MDMA in an enantiomeric excess relative to R(−)-MDMA, or a pharmaceutically acceptable salt thereof, and a pharmaceutically acceptable carrier; and (ii) a second composition comprising enantiomerically enriched MDMA comprising R(−)-MDMA in an enantiomeric excess relative to S(+)-MDMA, or a pharmaceutically acceptable salt thereof, and a pharmaceutically acceptable carrier. In embodiments of this aspect, the first composition comprises an amount of S(+)-MDMA that is effective to inhibit function and/or activity of CYP2D6. In this aspect, the second composition comprises the various R(−)-MDMA compositions (i.e., R(−)-MDMA in an enantiomeric excess relative to S(+)-MDMA) as described throughout the disclosure).

In embodiments, the first composition of the kit comprise racemic MDMA. In embodiments, the first composition of the kit comprise MDMA comprising S(+)-MDMA in an enantiomeric excess relative to R(−)-MDMA. For example, the kit comprise S(+)-MDMA in an enantiomeric excess relative to R(−)-MDMA in a total amount of about 50.01% to about 100% S(+)-MDMA. In embodiments, the amount of S(+)-MDMA in an enantiomeric excess relative to R(−)-MDMA may be from about 65% to about 100% S(+)-MDMA. In embodiments, the first composition of the kit comprise S(+)-MDMA in an enantiomerically pure form.

In embodiments, the first composition of the kit comprise MDMA comprising S(+)-MDMA in an enantiomeric enrichment (or purity) of ≥90%, ≥95%, ≥96%, ≥97%, ≥98%, ≥99%, ≥99.5%, ≥99.9%, ≥99.99%, or 100% (i.e., relative to total MDMA).

In embodiments wherein the first composition of the kit comprises MDMA comprising S(+)-MDMA in excess compared to R(−)-MDMA, the first composition comprises a weight ratio of S(+)-MDMA to R(−)-MDMA that is greater than 1 (e.g., about 50.1:49.9, 50.5:49.5, about 51:49, about 52:48, about 53:47, about 54:46, about 55:45, about 56:44, about 57:43, about 58:42, about 59:41, about 60:40, about 61:39, about 62:38, about 63:37, about 64:36, about 65:35, about 66:34, about 67:33, about 68:32, about 69:31, about 70:30, about 71:29, about 72:28, about 73:27, about 74:26, about 75:25, about 76:24, about 77:23, about 78:22, about 79:21, about 80:20, about 81:19, about 82:18, about 83:17, about 84:16, about 85:15, about 86:14, about 87:13, about 88:12, about 89:11, about 90:10, about 91:9, about 92:8, about 93:7, about 94:6, about 95:5, about 96:4, about 97:3, about 98:2, about 99:1, about 99.5:0.5, about 99.9:0.1, or about 100:0, among other ratios within these enumerated ratios).

In embodiments, the first composition of the kit comprise a weight ratio of S(+)-MDMA to R(−)-MDMA ranging from about 90:10 to about 100:0 (e.g., a weight ratio of S(+)-MDMA to R(−)-MDMA of about 90:10, about 91:9, about 92:8, about 93:7, about 94:6, about 95:5, about 96:4, about 97:3, about 98:2, about 99:1, about 99.5:0.5, about 99.9:0.1, or about 100:0. In embodiments, the first composition of the kit comprise a weight ratio of S(+)-MDMA to R(−)-MDMA ranging from about 99:1 to about 100:0. For example, the weight ratio of S(+)-MDMA to R(−)-MDMA comprises about 99.1:0.9, about 99.2:0.8, about 99.3:0.7, about 99.4:0.6, about 99.5:0.5, about 99.6:0.4, about 99.7:0.3, about 99.8:0.2, about 99.9:0.1, or about 100:0

As described above, a kit in accordance with the disclosure comprise a second composition comprising enantiomerically enriched MDMA comprising R(−)-MDMA in an enantiomeric excess relative to S(+)-MDMA, as described herein. For example, the second composition of the kit comprise R(−)-MDMA in an enantiomeric excess relative to S(+)-MDMA in a total amount of about 50.01% to about 100% S(+)-MDMA. In embodiments, the amount of R(−)-MDMA in an enantiomeric excess relative to S(+)-MDMA may be from about 65% to about 100% R(−)-MDMA. In embodiments, the amount of R(−)-MDMA in an enantiomeric excess relative to S(+)-MDMA may be from about 90% to about 100% R(−)-MDMA. In embodiments, the second composition of the kit comprise R(−)-MDMA in an enantiomerically pure form.

In embodiments, the second composition of the kit comprise enantiomerically enriched MDMA comprising R(−)-MDMA in an anantiomeric enrichment (or purity) of ≥90%, ≥95%, ≥96%, ≥97%, ≥98%, ≥99%, ≥99.5%, ≥99.9%, ≥99.99%, or 100% (i.e., relative to total MDMA).

In embodiments wherein the second composition of the kit comprises enantiomerically enriched MDMA comprising enantiomerically enriched R(−)-MDMA in excess compared to S(+)-MDMA, the second composition comprises a weight ratio of R(−)-MDMA to S(+)-MDMA that is greater than 1 (e.g., about 50.1:49.9, about 50.5:49.5, about 51:49, about 52:48, about 53:47, about 54:46, about 55:45, about 56:44, about 57:43, about 58:42, about 59:41, about 60:40, about 61:39, about 62:38, about 63:37, about 64:36, about 65:35, about 66:34, about 67:33, about 68:32, about 69:31, about 70:30, about 71:29, about 72:28, about 73:27, about 74:26, about 75:25, about 76:24, about 77:23, about 78:22, about 79:21, about 80:20, about 81:19, about 82:18, about 83:17, about 84:16, about 85:15, about 86:14, about 87:13, about 88:12, about 89:11, about 90:10, about 91:9, about 92:8, about 93:7, about 94:6, about 95:5, about 96:4, about 97:3, about 98:2, about 99:1, about 99.5:0.5, about 99.9:0.1, or about 100:0, among other ratios within these enumerated ratios).

In embodiments, the second composition of the kit comprise a weight ratio of R(−)-MDMA to S(+)-MDMA ranging from about 90:10 to about 100:0 (e.g., a weight ratio of R(−)-MDMA to S(+)-MDMA of about 90:10, about 91:9, about 92:8, about 93:7, about 94:6, about 95:5, about 96:4, about 97:3, about 98:2, about 99:1, about 99.5:0.5, about 99.9:0.1, or about 100:0. In embodiments, the second composition of the kit comprises a weight ratio of R(−)-MDMA to S(+)-MDMA ranging from about 99:1 to about 100:0. In embodiments, the weight ratio of R(−)-MDMA to S(+)-MDMA comprises about 99.1:0.9, about 99.2:0.8, about 99.3:0.7, about 99.4:0.6, about 99.5:0.5, about 99.6:0.4, about 99.7:0.3, about 99.8:0.2, about 99.9:0.1, or about 100:0.

In embodiments, the combined amount of S(+)-MDMA and R(−)-MDMA in the first composition of a kit in accordance with the disclosure may be effective to partially inhibit CYP2D6 metabolism of MDMA.

In embodiments, the kits of the present disclosure comprise a weight ratio of the first composition to the second composition that is about 1:1 or less than 1. In embodiments, the weight ratio of the first composition to the second composition ranges from about 1:1 to about 1:500 (e.g., about 1:2, about 1:5, about 1:10, about 1:20, about 1:30, about 1:40, about 1:50, about 1:60, about 1:70, about 1:80, about 1:90, about 1:100, about 1:110, about 1:120, about 1:130, about 1:140, about 1:150, about 1:160, about 1:170, about 1:180, about 1:190, about 1:200, about 1:210, about 1:220, about 1:230, about 1:240, about 1:250, about 1:260, about 1:270, about 1:280, about 1:290, about 1:300, about 1:310, about 1:320, about 1:330, about 1:340, about 1:350, about 1:360, about 1:370, about 1:380, about 1:390, about 1:400, about 1:410, about 1:420, about 1:430, about 1:440, about 1:450, about 1:460, about 1:470, about 1:480, about 1:490, about 1:500, about 1:510, about 1:520, about 1:530, about 1:540, about 1:550, about 1:560, about 1:570, about 1:580, about 1:590, about 1:600, about 1:610, about 1:620, about 1:630, about 1:640, about 1:650, about 1:660, about 1:1670, about 1:680, about 1:690, about 1:700, about 1:710, about 1:720, about 1:730, about 1:740, about 1:750, about 1:760, about 1:770, about 1:780, about 1:790, about 1:800, about 1:810, about 1:820, about 1:830, about 1:840, about 1:850, about 1:860, about 1:870, about 1:880, about 1:890, about 1:900, about 1:910, about 1:920, about 1:930, about 1:940, about 1:950, about 1:960, about 1:970, about 1:980, about 1:990, or about 1:1000 among other ratios within these enumerated ratios).

### Methods for Modulating MDMA Metabolism

MDMA metabolism includes two main metabolic pathways: (1) O-demethylation followed by catechol-O-methyltransferase (COMT)-catalyzed methylation and/or glucuronide/sulfate conjugation; and (2) N-dealkylation, deamination, and oxidation to the corresponding benzoic acid derivatives conjugated with glycine. The polymorphic enzyme CYP2D6 partially regulates the O-demethylation pathway of MDMA and yields the primary metabolite HHMA (3,4-dihydroxymethamphetamine). See, e.g., Pizarro N. et al., Drug Metabolism and Disposition. 2004, 32 (9): 1001-1007. The N-demethylation of MDMA to yield MDA occurs at a lower rate such that the overall metabolic fate of MDMA proceeds primarily through CYP2D6 mediated O-demethylation to yield HHMA.

In embodiments, the disclosure provides methods of perturbing the canonical metabolism of R(−)-MDMA to promote greater exposure of the active metabolite R(−)-MDA. Thus, in embodiments, the disclosure provides a method for sustaining responses to R(−)-MDA exposure in a subject comprising administering to the subject a composition, kit, or article of manufacture in accordance with the disclosure. CYP2D6 is a polymorphic enzyme with four general phenotypic categories: ultra-rapid, extensive (also referred to as efficient), intermediate, and poor metabolizers. In embodiments, the subject may have a CYP2D6 metabolic phenotype identified as ultra-rapid, extensive or efficient, or intermediate phenotype.

In embodiments, the disclosure provides methods of increasing or prolonging exposure of R(−)-MDA in plasma or brain of a subject in need thereof, the method comprising administering to the subject: a therapeutically effective amount of a CYP2D6 inhibitor in an amount effective to inhibit CYP2D6 metabolism of MDMA, and a therapeutically effective amount of enantiomerically enriched MDMA comprising R(−)-MDMA in an enantiomeric excess relative to S(+)-MDMA, or a pharmaceutically acceptable salt thereof. In embodiments, the CYP2D6 inhibitor may be administered before the enantiomerically enriched MDMA. In embodiments, the CYP2D6 inhibitor may be administered at the same time as the enantiomerically enriched MDMA.

As described above, compositions, kits, and articles of manufacture in accordance with the disclosure comprise various weight ratios of the inhibitor to CYP2D6 to enantiomerically enriched MDMA that is 1:1 or less than 1. Thus, in embodiments, methods in accordance with the disclosure of increasing or prolonging exposure to R(−)-MDA in plasma or brain of a subject in need thereof comprise administering to the subject CYP2D6 and enantiomerically enriched MDMA in a weight ratio ranging from about 1:1 to about 1:500 (among other ratios described above). In embodiments, compositions, kits, and articles of manufacture in accordance with the disclosure comprises a weight ratio of the inhibitor to CYP2D6 to enantiomerically enriched MDMA from about 1:1 to about 1:25.

In embodiments, the inhibitor of CYP2D6 aministered to a subject in accordance with methods of increasing or prolonging exposure of R(−)-MDA comprise a reversible CYP2D6 inhibitor. In embodiments, the reversible inhibitor of CYP2D6 comprises quinidine, bupropion, fluoxetine, terbinafine, perhexiline, abiraterone, cinacalcet, duloxetine, lorcaserin, mirabegron, amiodarone, celecoxib, clobazam, cobicistat, desvenlafaxine, diltiazem, diphenhydramine, Echinacea, escitalopram, febuxostat, fluvoxamine, gefitinib, hydralazine, hydroxychloroquine, imatinib, labetalol, methadone, pazopanib, propafenone, ranitidine, ritonavir, sertraline, telithromycin, verapamil, vemurafenib, terfenadine, progesterone, testosterone, lansoprazole, olanzapine, chlorpromazine, fluphenazine, haloperidol, thioridazine, risperidone, clozapine, trifluperidol, Panax ginseng, or Ginko biloba. In embodiments, the reversible inhibitor of CYP2D6 comprises quinidine, terbinafine, perhexiline, abiraterone, cinacalcet, duloxetine, lorcaserin, mirabegron, amiodarone, celecoxib, clobazam, cobicistat, desvenlafaxine, diltiazem, diphenhydramine, Echinacea, febuxostat, fluvoxamine, gefitinib, hydralazine, hydroxychloroquine, imatinib, labetalol, methadone, pazopanib, propafenone, ranitidine, ritonavir, sertraline, telithromycin, verapamil, vemurafenib, terfenadine, progesterone, testosterone, lansoprazole, olanzapine, chlorpromazine, fluphenazine, haloperidol, thioridazine, risperidone, clozapine, trifluperidol, Panax ginseng, or Ginko biloba. In embodiments, the reversible inhibitor of CYP2D6 administered to a subject is not fluoxetine, sertraline, escitalopram, or bupropion.

In embodiments, the inhibititor of CYP2D6 administered to a subject in accordance with methods of increasing or prolonging exposure of R(−)-MDA comprise an irreversible CYP2D6 inhibitor. In embodiments, the irreversible CYP2D6 inhibitor comprise paroxetine, cimetidine, pimozide, methamphetamine, metoclopramide, or desethylamiodarone. In embodiments, the irreversible inhibitor of CYP2D6 administered to a subject is not paroxetine.

In embodiments of the methods described herein, the method comprises increasing exposure of R(−)-MDA in plasma or brain, as compared to a subject administered the same dose of enantiomerically enriched MDMA without administration of a CYP2D6 inhibitor in an amount effective to inhibit CYP2D6 metabolism of MDMA. In embodiments, the exposoure of R(−)-MDA in plasma or brain is increased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, about 120%, about 125%, about 130%, about 135%, about 140%, about 145%, about 150%, about 155%, about 160%, about 165%, about 170%, about 175%, about 180%, about 185%, about 190%, about 195%, about 200%, including all values and ranges therein.

In embodiments of the methods described herein, the method comprises prolonging exposoure of R(−)-MDA in plasma or brain, as compared to a subject administered the same dose of enantiomerically enriched MDMA without administration of a CYP2D6 inhibitor in an amount effective to inhibit CYP2D6 metabolism of MDMA. In embodiments, the exposoure of R(−)-MDA in plasma or brain is prolonged by about 0.5 hour, about 0.75 hour, about 1 hour, about 1.25 hours, about 1.5 hours, about 1.75 hours, about 2.0 hours, about 2.25 hours, about 2.5 hours, about 2.75 hours, about 3.0 hours, about 3.25 hours, about 3.5 hours, about 3.75 hours, about 4.0 hours, about 4.25 hours, about 4.5 hours, about 4.75 hours, about 5.0 hours, about 5.25 hours, about 5.5 hours, about 5.75 hours, about 6.0 hours, about 6.25 hours, about 6.5 hours, about 6.75 hours, about 7.0 hours, about 7.25 hours, about 7.5 hours, about 7.75 hours, about 8.0 hours 8.25 hours, about 8.5 hours, about 8.75 hours, about 9.0 hours, about 9.25 hours, about 9.5 hours, about 9.75 hours, about 10.0 hours, about 10.25 hours, about 10.5 hours, about 10.75 hours, about 11.0 hours, about 11.25 hours, about 11.5 hours, about 11.75 hours, about 12.0 hours, about 12.25 hours, about 12.5 hours, about 12.75 hours, about 13.0 hours, about 13.25 hours, about 13.5 hours, about 13.75 hours, about 14.0 hours, about 14.25 hours, about 14.5 hours, about 14.75 hours, about 15.0 hours, about 15.25 hours, about 15.5 hours, about 15.75 hours, about 16.0 hours, about 16.25 hours, about 16.5 hours, about 16.75 hours, about 17.0 hours, about 17.25 hours, about 17.5 hours, about 17.75 hours, about 18.0 hours 18.25 hours, about 18.5 hours, about 18.75 hours, about 19.0 hours, about 19.25 hours, about 19.5 hours, about 19.75 hours, about 20.0 hours, about 20.25 hours, about 20.5 hours, about 20.75 hours, about 21.0 hours, about 21.25 hours, about 21.5 hours, about 21.75 hours, about 22.0 hours, about 22.25 hours, about 22.5 hours, about 22.75 hours, about 23.0 hours, about 23.25 hours, about 23.5 hours, about 23.75 hours, or about 24.0 hours or more including all values and ranges therein.

The disclosure additionally provides methods increasing or prolonging exposure to R(−)-MDA in plasma or brain of a subject in need thereof comprising administering to the subject a first dose of MDMA comprising S(+)-MDMA, in an amount effective to inhibit CYP2D6 metabolism of MDMA,or a pharmaceutically acceptable salt thereof, and a second dose of enantiomerically enriched MDMA comprising R(−)-MDMA in an enantiomeric excess relative to S(+)-MDMA, or a pharmaceutically acceptable salt thereof. In embodiments, the first dose of MDMA comprising S(+)-MDMA may be racemic MDMA. In embodiments, the first dose of MDMA comprising S(+)-MDMA may be MDMA enantiomerically enriched in S(+)-MDMA (i.e., wherein the weight ratio of S(+)-MDMA to R(−)-MDMA is greater than 1).

As described above, compositions, kits, and articles of manufacture in accordance with the disclosure comprise R(−)-MDMA in enantiomeric excess relative to S(+)-MDMA at various weight ratios. Thus, in embodiments, methods in accordance with the disclosure comprise an amount of R(−)-MDMA in enantiomeric excess relative to S(+)-MDMA that may be from about 50.01% to about 100% R(−)-MDMA (i.e., a weight ratio that is greater than 1). Thus, in embodiments, methods in accordance with the disclosure of increasing or prolonging exposure of R(−)-MDA in plasma or brain of a subject in need thereof comprise administering to the subject a second dose of MDMA comprising R(−)-MDMA that comprises at least about 50.01% to 100% of the total amount of MDMA. In embodiments, the R(−)-MDMA comprise about 90% to about 100% of the total amount of MDMA. In embodiments, 100% R(−)-MDMA comprise an enantiomerically pure form of R(−)-MDMA as measured by chiral HPLC.

In embodiments of the methods described herein, the method comprises increasing exposoure of R(−)-MDA in plasma or brain, as compared to a subject administered the same dose of enantiomerically enriched MDMA comprising R(−)-MDMA in an enantiomeric excess relative to S(+)-MDMA without administration of MDMA comprising S(+)-MDMA in an amount effective to inhibit CYP2D6 metabolism of MDMA. In embodiments, the exposoure of R(−)-MDA in plasma or brain is increased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, about 120%, about 125%, about 130%, about 135%, about 140%, about 145%, about 150%, about 155%, about 160%, about 165%, about 170%, about 175%, about 180%, about 185%, about 190%, about 195%, about 200%, including all values and ranges therein.

In embodiments of the methods described herein, the method comprises prolonging exposure of R(−)-MDA in plasma or brain, as compared to a subject administered the same dose of enantiomerically enriched MDMA comprising R(−)-MDMA in an enantiomeric excess relative to S(+)-MDMA without administration of MDMA comprising S(+)-MDMA in an amount effective to inhibit CYP2D6 metabolism of MDMA. In embodiments, the exposure of R(−)-MDA in plasma or brain is prolonged by about 0.5 hour, about 0.75 hour, about 1 hour, about 1.25 hours, about 1.5 hours, about 1.75 hours, about 2.0 hours, about 2.25 hours, about 2.5 hours, about 2.75 hours, about 3.0 hours, about 3.25 hours, about 3.5 hours, about 3.75 hours, about 4.0 hours, about 4.25 hours, about 4.5 hours, about 4.75 hours, about 5.0 hours, about 5.25 hours, about 5.5 hours, about 5.75 hours, about 6.0 hours, about 6.25 hours, about 6.5 hours, about 6.75 hours, about 7.0 hours, about 7.25 hours, about 7.5 hours, about 7.75 hours, about 8.0 hours 8.25 hours, about 8.5 hours, about 8.75 hours, about 9.0 hours, about 9.25 hours, about 9.5 hours, about 9.75 hours, about 10.0 hours, about 10.25 hours, about 10.5 hours, about 10.75 hours, about 11.0 hours, about 11.25 hours, about 11.5 hours, about 11.75 hours, about 12.0 hours, about 12.25 hours, about 12.5 hours, about 12.75 hours, about 13.0 hours, about 13.25 hours, about 13.5 hours, about 13.75 hours, about 14.0 hours, about 14.25 hours, about 14.5 hours, about 14.75 hours, about 15.0 hours, about 15.25 hours, about 15.5 hours, about 15.75 hours, about 16.0 hours, about 16.25 hours, about 16.5 hours, about 16.75 hours, about 17.0 hours, about 17.25 hours, about 17.5 hours, about 17.75 hours, about 18.0 hours 18.25 hours, about 18.5 hours, about 18.75 hours, about 19.0 hours, about 19.25 hours, about 19.5 hours, about 19.75 hours, about 20.0 hours, about 20.25 hours, about 20.5 hours, about 20.75 hours, about 21.0 hours, about 21.25 hours, about 21.5 hours, about 21.75 hours, about 22.0 hours, about 22.25 hours, about 22.5 hours, about 22.75 hours, about 23.0 hours, about 23.25 hours, about 23.5 hours, about 23.75 hours, or about 24.0 hours or more including all values and ranges therein.

### Methods of Use

In aspects, the compositions and kits of the present disclosure is used to treat a neurological disease, disorder, or condition, (e.g., a mental health disease, disorder, or condition). In embodiments, provided herein are methods of treating a neurological disease, disorder, or condition comprising administering to a patient in need thereof any of the compositions and kits described herein.

As referred to herein, a “neurological disease disorder, or condition” refers to a disease, disorder, or condition comprising: a neuropsychiatric disorder, such as depression (including severe depression such as treatment-resistant depression (TRD), major depressive disorder (MDD) and persistent depressive disorder), alexithymia, catatonic depression, a depressive disorder due to a medical condition, postpartum depression, premenstrual dysphoric disorder, seasonal affective disorder, anxiety, anxiety disorder, social anxiety disorder (SAD), general anxiety disorder (GAD), avolition disorder, bipolar disorder (including bipolar I disorder and bipolar II disorder), post-traumatic stress disorder (PTSD), body dysmorphic disorder, abnormalities of mood or emotion, including the above conditions, dysthymia, schizoaffective disorder, schizophrenia and other psychotic disorders, panic disorder, traumatic stress disorders, phobic disorders, and personality disorders with abnormal mood, such as borderline personality disorder, schizoid and schizotypal disorders, suicide ideation, rumination/unproductive repetitive thoughts negatively impacting one's behavior, mood, and/or ability to focus, obsessive-compulsive disorder, addiction (including substance use disorders, such as addiction to nicotine, alcohol, cocaine, opioids, amphetamine, methamphetamine, heroin, morphine, phencyclidine, 3,4-methylenedioxy-methamphetamine, as well as other addictive substances), addictive behavior (including eating, gambling, sex, pornography, videogames, work, exercise, spiritual obsession, self-harm, travel and shopping addiction, etc.), eating disorders (including anorexia nervosa, bulimia nervosa and binge eating disorder), pain (including pain associated with migraine or headache or chronic pain), Prolonged Grief Disorder, paranoid personality disorder, Autoimmune OCD (adult), behavioral symptoms/anxiety in Fragile X (adult), or Xenomelia.

In embodiments, the disclosure provides the use of any of the compositions and kits described herein for treating or alleviating one or more neurological diseases, disorders, or conditions. In embodiments, the neurological disease, disorder, or condition comprises a mental health disease, disorder, or condition. Such conditions including those identified above and herein are described and discussed in the Diagnostic and Statistical Manual of Mental Disorders (5^{th} ed.; DSM-5; American Psychiatric Association, 2013), the content of which is incorporated by reference in its entirety for any purpose.

In embodiments, the disclosure provides methods for the treatment of depression, substance use disorders (SUD), and/or eating disorders. In embodiments, the disclosure provides methods for the treatment of an eating or feeding disorder, such as, for example anorexia nervosa, bulimia nervosa, binge eating, and/or other disorders related to eating.

In embodiments, the disclosure provides methods for the treatment of a mood disorder such as, for example, depressive disorders, such as major depressive disorder or treatment resistant depression.

In embodiments, the disclosure provides methods for the treatment of a dissociative disorder such as depersonalization-derealization disorder.

In embodiments, the disclosure provides methods for the treatment of a somatic symptom disorder and related disorders.

In embodiments, the disclosure provides methods for the treatment of a condition relating to sexual dysfunction such as, for example, female orgasmic disorder, female sexual interest disorder, female arousal disorder, and/or male hypoactive sexual desire disorder.

In embodiments, the disclosure provides methods for the treatment of a personality disorder, such as disorders classified as Cluster A, Cluster B, and/or Cluster C disorders which include, for example, adjustment disorder, antisocial personality disorder, borderline personality disorder, histrionic personality disorder and/or avoidant personality disorder.

In embodiments, the disclosure provides methods for the treatment of a depressive episode with short-duration hypomania.

In embodiments for treating a substance abuse disorder (SUD) provided herein, substance use related disorders are typically disorders of maladaptive patterns of substance use, and include criteria, such as recurrent substance use related problems, tolerance to a substance, withdrawal upon discontinuing use, an inability to cut down or control use of the substance, and giving up important social, occupational, or recreational activities because of using the substance. See e.g., DSM-5. In embodiments, the substance use related disorder is a disorder resulting from the use of: alcohol; caffeine; cannabis; hallucinogens (such as phencyclidine or similarly acting arylcyclohexylamines, and other hallucinogens, such as LSD); inhalants; opioids; sedatives, hypnotics, or anxiolytics; stimulants (including amphetamine-type substances, cocaine, and other stimulants); tobacco; and other substances.

In embodiments, the disclosure provides methods for the treatment of one or more conditions associated with anosognosia (e.g., denial of deficit or lack of insight), borderline personality disorder, bereavement, rejection sensitivity (e.g., as associated with attention deficit disorders and/or hyperactivity disorders), and Cluster C personality disorders (e.g., avoidant personality disorder, dependent personality disorder, and/or obsessive-compulsive personality disorder), which are typically characterized as including a consistently dysfunctional pattern of anxious thinking or behavior.

A subject who has develops, or is suspected of having or developing, a Cluster C personality disorder can be diagnosed, identified, and/or characterized as also exhibiting symptoms of at least one other type of Cluster C disorder. As such, the number of symptoms exhibited by a subject may vary widely.

Avoidant personality disorder can be associated with symptoms such as, for example, high or increased sensitivity to criticism or rejection; feelings of inadequacy, lack of importance, and/or unattractive; isolation; withdrawn from activities and/or social interaction; fear of disapproval; fear of embarrassment; and/or a target of criticism.

Dependent personality disorder can be associated with symptoms such as, for example, a lack of self-reliance; overly-reliant on others; being submissive and/or clingy toward others; fear of abandonment; lack of self-confidence; inability to make decisions without input from others; inability to motivate or self-start; seeking approval from others; endures abuse or poor treatment from others; and/or relationship seeking.

Obsessive-compulsive personality disorder (different from obsessive-compulsive disorder, which is an anxiety-based disorder) can be associated with symptoms such as, for example, over-emphasis on details, orderliness and/or rules; seeking perfection; controlling of people, tasks, money, situations and circumstances; inability to delegate tasks; rigidity in attitude; neglecting relationships (i.e., favoring work over friends); stubbornness; and/or inflexibility in attitude toward alternative opinions and/or perspectives.

Borderline personality disorder can be associated with symptoms such as, for example, difficulty processing or managing emotion, self-image issues, highly variable, quick, intense, and/or extreme mood and/or behavioral changes, impulsive behavior (spending, promiscuity, binge eating and/or drinking, recklessness, etc.), self-harming behavior (suicidal thoughts, threats, and/or attempts), avoidance of abandonment, intense and/or unstable relationships, lack of trusting in others, feelings of emptiness, inability to manage anger, and/or feelings of dissociation (e.g., dissociated from one's body).

In embodiments of methods for treating a subject who has or develops a disease, disorder, or condition that is associated with anosognosia (lack of insight or denial of deficit), the methods and uses can treat a subject who is unable to recognize the underlying illness or condition and/or may not seek treatment or maintain an existing treatment. Anosognosia can be associated with many mental health disorders, but is most often associated in subjects who have or develop schizophrenia, dementia, Alzheimer's disease, and bipolar disorder, as non-limiting examples. Non-limiting symptoms of anosognosia include an inability to: recognize one's own illness or medical problem; recognize the signs and symptoms of the condition being experienced; to connect signs and symptoms being experienced with the underlying disorder or condition; and/or understand or agree that treatment is needed for the underlying disorder or that the condition is serious.

In embodiments of methods for treating adjustment disorders, the disorder can be characterized by the development of emotional and/or behavioral symptoms in response to an identifiable stressor within three months of symptom onset. The type of stressor may vary in significance for children and adolescents relative to adults. The symptoms or behaviors are usually clinically significant, typically including one or both of: (i) marked distress that is out of proportion to the severity or intensity of the stressor (given external context and the cultural factors that might influence symptom severity and presentation); and (ii) significant impairment in social, occupational, and/or other important areas of functioning. Adjustment disorders are typically triggered by a stress-related disturbance that does not meet any criteria for other mental disorders, or a heightening of a preexisting condition. The symptoms associated with an adjustment disorder are also typically not characteristic of normal bereavement. In addition, once the stressor has ended, the symptoms do not persist for more than an additional six months. Adjustment disorders can be characterized as acute (lasting less than six months) or chronic (lasting more than six months). Typically adjustment disorders can be identified by one or more of six general subtypes, including (a) depressed mood; (b) anxiety; (c) mixed anxiety and depressed mood; (d) disturbance of conduct; (e) mixed disturbance of emotions and conduct; and (f) unspecified (not classifiable as one of the specific subtypes (a)-(e)). These six types of adjustment disorders can be characterized by several symptoms such as those described below.

Adjustment disorder with depressed mood: Symptoms can include feeling sad, tearful and hopeless, very tired, and taking no pleasure in the things the subject used to enjoy.

Adjustment disorder with anxiety: Symptoms can include nervousness, worry, having a hard time concentrating or remembering things, and/or feeling overwhelmed. Children may strongly fear being separated from their parents and loved ones.

Adjustment disorder with mixed anxiety and depressed mood: Symptoms can include a blend of depression and anxiety.

Adjustment disorder with disturbed conduct: Symptoms can include behavioral problems, such as fighting or reckless driving. Children and teenagers may skip school or damage or destroy property.

Adjustment disorder disturbed emotions and conduct: Symptoms can include a mix of depression, anxiety and behavioral problems.

Unspecified adjustment disorder: Symptoms do not fit well within the other types of adjustment disorders, but can include physical problems, problems in relationships with family or friends, or problems at work or school

Anxiety disorders include panic disorder, agoraphobia with or without history of panic disorder, specific phobia, social phobia, obsessive-compulsive disorder, post-traumatic stress disorder, acute stress disorder and generalized anxiety disorder. In embodiments, the compositions and kits described herein can be used to treat these disorders in patients who have been diagnosed as having such disorders. In embodiments, the disclosure provides methods for treating a patient suffering from anxiety which comprises administering to the patient an amount of any of the pharmaceutical compositions or kits described herein effective to treat the subject's anxiety.

Social anxiety disorder (SAD), also known as social phobia, is characterized by a marked and persistent fear of one or more social or performance situations in which the person is exposed to unfamiliar people or to possible scrutiny by others (American Psychiatric Association, 1994a). Exposure to the feared situation almost invariably provokes anxiety, which may approach the intensity of a panic attack. The feared situations are avoided or endured with intense anxiety or distress. The avoidance, anxious anticipation, or distress in the feared situation(s) interferes significantly with the person's normal routine, occupational or academic functioning, or social activities or relationships, or there is marked distress about having the phobias. Lesser degrees of performance anxiety or shyness generally do not require psychopharmacological treatment.

In embodiments, the compositions and kits described herein is used to treat social anxiety disorder in patients who have been diagnosed as having social anxiety disorder based upon the administration of any of the following tests: Liebowitz Social Anxiety Scale (LSAS), CGI-Severity of Illness scale, Social Phobia Inventory (SPIN) total score, social Connectedness Scale, Internalized Shame Scale, Quick Inventory of Depressive Symptomatology (QIDS), Columbia Suicide Severity Rating Scale, Self-Compassion Scale (SCS) total score, Psychotomimetic States Inventory (PSI), Challenging Experience Questionnaire (CEQ) total score, Emotional Breakthrough Inventory (EBI) total score, Mystical Experience Questionnaire, EQL-5D, Hamilton Rating Scale for Anxiety (HAM-A), Hamilton Rating Scale for Depression (HAM-D), axis V Social and Occupational Functioning Assessment Scale of DSM-IV, axis II (ICD-10) World Health Organization Disability Assessment, Schedule 2 (DAS-2), Sheehan Disability Scales, Schneier Disability Profile, World Health Organization Quality of Life-100 (WHOQOL-100), Quality of Life Inventory (QOLI), European Quality of Life 5-Dimensions scale or other tests as described in Bobes, J. (1998), How is recovery from social anxiety disorder defined? The Journal of Clinical Psychiatry, 59(Suppl 17), 12-19, which is incorporated herein by reference. In embodiments, the compositions and kits described herein is used to induce improvements as measured by these tests, such as the a change from baseline in the Liebowitz Social Anxiety Scale (LSAS), or a CGI-Global Improvement score of 1 (very much improved), 2 (much improved) or 3 (minimally improved). In embodiments, the compositions and kits described herein is used to prevent relapse of social anxiety disorder. In embodiments, the disclosure provides methods of treating social anxiety disorder in a patient which comprises administering to the patient a therapeutically effective amount of any of the compositions or kits utilized herein to treat the subject's social anxiety disorder.

In embodiments, the compositions and kits described herein are used to treat obsessions and compulsions in patients who have been diagnosed as having obsessive compulsive disorder based upon administration of appropriate tests, which may include, but are not limited to any of the following: Yale Brown Obsessive Compulsive Scale (YBOCS) (for adults), National Institute of Mental Health Global OCD Scale (NIMH GOCS), CGI-Severity of Illness scale. It is further contemplated that the compounds described herein will be effective in inducing improvements in certain of the factors measured in these tests, such as a reduction of several points in the YBOCS total score. In embodiments, the compositions and kits described herein are used to prevent relapse of obsessive compulsive disorder and/or symptoms thereof. In embodiments, the disclosure provides methods of treating obsessions and/or compulsions in a patient with obsessive compulsive disorder, which comprises administering to the patient a therapeutically effective amount of any of the compositions and kits utilized herein to treat the subject's obsessions and/or compulsions.

In embodiments, the compositions and kits described herein are used to treat panic disorder in patients who have been diagnosed with panic disorder on the basis of frequency of occurrence of panic attacks, or by means of the CGI-Severity of Illness scale. In embodiments, the compositions and kits described herein are used to induce improvements in certain of the factors measured in these evaluations, such as a reduction in frequency or elimination of panic attacks, an improvement in the CGI-Severity of Illness scale or a CGI-Global Improvement score of 1 (very much improved), 2 (much improved) or 3 (minimally improved). In embodiments, the compositions and kits described herein are used to prevent relapse of panic disorder. In embodiments, the disclosure provides methods of treating panic disorder, with or without agoraphobia, in a subject, which comprises administering to the patient a therapeutically effective amount of any of the compositions and kits utilized herein to treat the subject's panic disorder.

In embodiments, the compositions and kits described herein are used to treat generalized anxiety disorder in patients who have been diagnosed as having this disorder based upon the diagnostic criteria described in DSM-5. In embodiments, the compositions and kits described herein are used to reduce symptoms of this disorder, such as the following: excessive worry and anxiety, difficulty controlling worry, restlessness or feeling keyed up or on edge, being easily fatigued, difficulty concentrating or mind going blank, irritability, muscle tension, or sleep disturbance. In embodiments, the compositions and kits described herein are used to prevent relapse of general anxiety disorder. In embodiments, the disclosure provides methods of treating generalized anxiety disorder in a subject, which comprises administering to the patient an amount of any of the compositions and kits described herein to treat the subject's generalized anxiety disorder.

In embodiments, the compositions and kits described herein are used to treat impulse control disorders in patients who have at least one impulse control disorder based upon the diagnostic criteria described in DSM-5. Impulse control disorders include pathological gambling (PG), kleptomania, trichotillomania (TTM), intermittent explosive disorder (IED), and pyromania. Impulse control disorders may also include pathological skin picking (PSP), compulsive sexual behavior (CSB), compulsive buying (CB), conduct disorder, antisocial personality disorder, oppositional defiant disorder, borderline personality disorder, attention deficit/hyperactivity disorder (ADHD, which includes attention deficit disorder, ADD), schizophrenia, mood disorders, paraphilia, and internet addiction. Symptoms of impulse control disorders include: repetitive participation in behavior despite adverse consequences, diminished control over the behavior, an urge/impulse to engage in the behavior, and feelings of pleasure while participating in the behavior. In embodiments, the compositions and kits described herein are used to reduce symptoms of this disorder, including impulsivity or lack of self-control. In embodiments, the compositions and kits described herein are used to prevent relapse of the impulse control disorder. In embodiments, the disclosure provides methods for treating impulse control disorders in a subject, which comprises administering to the patient a therapeutically effective amount of any of the compositions and kits utilized herein to treat the subject's impulse control disorders.

In embodiments, the compositions and kits described herein are used to treat ADHD or ADD in patients who have the disorder, based upon the diagnostic criteria described in DSM-5. In embodiments, the compositions and kits described herein are used to reduce symptoms of this disorder, including impulsivity or lack of self-control. In embodiments, the compositions and kits described herein are used to prevent relapse of ADD or ADHD. In embodiments, the disclosure provides methods for treating ADHD or ADD in a subject, which comprises administering to the patient a therapeutically effective amount of any of the compositions and kits utilized herein to treat the subject's ADHD or ADD.

In embodiments, the compositions and kits described herein are used to treat schizophrenia in patients who have the disorder, based upon the diagnostic criteria described in DSM-5. Schizophrenia is characterized by delusions, hallucinations, disorganized speech and behavior, and other symptoms that cause social or occupational dysfunction. In embodiments, the compositions and kits described herein are used to reduce symptoms of this disorder. In embodiments, the compositions and kits described herein are used to prevent relapse of schizophrenia. In embodiments, the disclosure provides methods for treating non-schizophrenia in a subject, which comprises administering to the patient a therapeutically effective amount of any of the compositions and kits utilized herein to treat the subject's schizophrenia.

In embodiments, the compositions and kits described herein are used to treat non-suicidal self injury disorder in patients who have been diagnosed with this disorder based on the patient's exhibition of symptoms including deliberate tissue injury without suicidal intent (e.g., cutting, burning, self-poisoning, or self-mutilation). In embodiments, the compositions and kits described herein are used to reduce improvements in certain of these factors, such as a reduction in frequency or elimination of self injury. In embodiments, the compositions and kits described herein are used to prevent relapse of non-suicidal self injury disorder. In embodiments, the disclosure provides methods for treating non-suicidal self injury disorder in a subject, which comprises administering to the patient a therapeutically effective amount of any of the compositions and kits utilized herein to treat the subject's non-suicidal self injury disorder.

In embodiments, the compositions and kits described herein are used to treat Münchausen syndrome in patients who have been diagnosed with this disorder based on the patient's propensity for feigning disease, illness, or psychological trauma to draw attention, sympathy, or reassurance to themselves. Symptoms may include frequent hospitalizations, knowledge of several illnesses, frequent requests for medication (e.g., pain killers), willingness to undergo extensive surgery, few to no visitors during hospitalizations, and exaggerated or fabricated stories about multiple medical problems. In embodiments, the compositions and kits described herein are used to induce improvements in certain of these factors, such as a reduction in frequency or elimination of one or more symptoms. In embodiments, the compositions and kits described herein are used to prevent relapse of Münchausen syndrome. Münchausen syndrome also includes Münchausen syndrome by proxy, in which a caregiver exaggerates, fabricates, or induces illness in someone in his/her care. In embodiments, the disclosure provides methods for treating Münchausen syndrome in a subject, which comprises administering to the patient a therapeutically effective amount of any of the compositions and kits utilized herein to treat the subject's Münchausen syndrome.

In embodiments, the compositions and kits described herein are used to treat disruptive mood dysregulation disorder in patients who have been diagnosed with this disorder on the basis of severe and recurrent temper outbursts, grossly out of proportion to the stimulus or situation, as well as a persistent irritable/angry mood most of the time. In embodiments, the compositions and kits described herein are used to induce improvements in certain of these factors, such as a reduction in frequency or elimination of tember outbursts and/or an improvement in mood. In embodiments, the compositions and kits described herein are used to prevent relapse of disruptive mood dysregulation disorder disorder. In embodiments, the disclosure provides methods for treating disruptive mood dysregulation disorder in a subject, which comprises administering to the patient a therapeutically effective amount of any of the compositions and kits utilized herein to treat the subject's disruptive mood dysregulation disorder.

In embodiments, the compositions and kits described herein are used to reduce the frequency, intensity, and duration of anger and/or violence in individuals. Although anger and violence disorders other than those associated with other disorders (e.g., as described above) are not outlined in DSM 5, many health professionals recognize that such disorders are associated with significant dysfunction. Anger management training and other psychosocial treatments are often used in an effort to treat these individuals. In embodiments, the disclosure provides methods for treating anger-and/or violence-related disorder in a subject, which comprises administering to the patient a therapeutically effective amount of any of the compositions and kits utilized herein to treat the subject's anger-and/or violence-related disorder.

In embodiments, the compositions and kits described herein are used to regulate food intake and/or reducing food cravings in patients in need thereof. In embodiments, the patient is overweight. In embodiments, the patient is obese. In embodiments, the patient exhibits comorbidities associated with overweight/obesity, for example coronary heart disease, high blood pressure, stroke, type 2 diabetes, abnormal levels of blood fats, metabolic syndrome, cancer, osteoarthritis, sleep apnea, reproductive issues, and/or gallstones. In embodiments, the disclosure provides methods of regulating food intake and/or reducing food cravings in a subject, which comprises administering to the patient a therapeutically effective amount of any of the compositions and kits utilized herein to regulate/reduce the subject's food intake and/or food cravings.

In embodiments, the disclosed methods are effective to treat one or more disease, disorder, or condition that is characterized by one or more symptoms. In embodiments, the one or more symptoms are one or more transdiagnostic symptom(s) that are associated with more than one disease, disorder, or condition. In embodiments, the method in accordance with the disclosure can treat the underlying disease, disorder, or condition and/or treat the transdiagnostic symptom(s) (e.g., reducing severity, intensity, frequency of the symptom(s), blocking the symptom(s), slowing progression or escalation of the symptom(s), reducing or eliminating recurrence of the symptom(s), inhibiting the symptom(s), or ameliorating the symptom(s)). In embodiments, the transdiagnostic symptom comprises any one or more of symptoms including, for example, one or more symptoms associated with a general increase associated with rejection sensitivity, emotional regulation, anhedonia, self-doubt/criticism, and/or lack of insight. In embodiments, the one or more transdiagnostic symptom comprises cognitive inflexibility or a lack of ability to shift perspective, negative emotional attention bias, a tendency to focus attention on negatively-valanced emotions and/or events, cognitive dysregulation, intrusive thoughts, restricted empathy for ones-self, restricted empathy for others, rigid thinking, non-response to psychotherapy, distorted cognitions, inappropriate responses, negative self-appraisal, negative feelings of self-worth, anxiety, rumination, depressed mood, negative cognitions, loss of ability to feel pleasure, social withdrawal or avoidance, problems with interpersonal relationships, diminished pleasure, loss of libido, emotional bluntness, lack of emotional regulation, irritability, anger, mood swings, intense outburst, aggressive outburst, self-harm, suicidal ideation, substance abuse, lack of impulse control, and/or fear from an expectation of rejection (whether real or imagined).

### Dosage Regimens

In embodiments of compositions and kits comprsing enantiomerically enriched MDMA comprising R(−)-MDMA in an enantiomeric excess relative to S(+)-MDMA and an inhibitor of CYP2D6, the therapeutically effective amount of the enantiomerically enriched MDMA to be administered in accordance with the methods described herein comprises a dose ranging from about 1 mg to about 1000 mg MDMA, or an equivalent dose of the pharmaceutically acceptable salt thereof, based on a 60 kg mammal subject (e.g., a human subject). In embodiments, the therapeutically effective amount comprises a dose ranging from about 1 mg to about 750 mg MDMA, or an equivalent dose of the pharmaceutically acceptable salt thereof. In embodiments, the therapeutically effective amount comprises a dose ranging from about 5 mg to about 500 mg, or about 10 mg to about 400 mg, or about 25 mg to about 300 mg, or about 75 mg to about 225 mg, or about 75 mg to about 850 mg, or about 250 mg to about 850 mg, or about 250 mg to about 350 mg MDMA, or an equivalent dose of the pharmaceutically acceptable salt thereof. In embodiments, the therapeutically effective amount comprises a dose ranging from about 25 mg to 50 mg MDMA, or an equivalent dose of the pharmaceutically acceptable salt thereof (e.g., about 20 mg, about 15 mg, about 10 mg, about 5 mg, about 1 mg, about 0.1 mg, about 0.01 mg, or about 0.001 mg). In embodiments, the therapeutically effective amount comprises a dose of about 25mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, about 120 mg, about 121 mg, about 122 mg, about 123 mg, about 124 mg, about 125 mg, about 126 mg, about 127 mg, about 128 mg, about 129 mg, about 130 mg, about 131 mg, about 132 mg, about 133 mg, about 134 mg, about 135 mg, about 136 mg, about 137 mg, about 138 mg, about 139 mg, about 140 mg, about 141 mg, about 142 mg, about 143 mg, about 144 mg, about 145 mg, about 146 mg, about 147 mg, about 148 mg, about 149 mg, about 150 mg, about 151 mg, about 152 mg, about 153 mg, about 154 mg, about 155 mg, about 156 mg, about 157 mg, about 158 mg, about 159 mg, about 160 mg, about 161 mg, about 162 mg, about 163 mg, about 164 mg, about 165 mg, about 166 mg, about 167 mg, about 168 mg, about 169 mg, about 170 mg, about 171 mg, about 172 mg, about 173 mg, about 174 mg, about 175 mg, about 176 mg, about 177 mg, about 178 mg, about 179 mg, about 180 mg, about 181 mg, about 182 mg, about 183 mg, about 184 mg, about 185 mg, about 186 mg, about 187 mg, about 188 mg, about 189 mg, about 190 mg, about 191 mg, about 192 mg, about 193 mg, about 194 mg, about 195 mg, about 196 mg, about 197 mg, about 198 mg, about 199 mg, about 200 mg, about 201 mg, about 202 mg, about 203 mg, about 204 mg, about 205 mg, about 206 mg, about 207 mg, about 208 mg, about 209 mg, about 210 mg, about 211 mg, about 212 mg, about 213 mg, about 214 mg, about 215 mg, about 216 mg, about 217 mg, about 218 mg, about 219 mg, about 220 mg, about 221 mg, about 222 mg, about 223 mg, about 224 mg, about 225 mg, about 226 mg, about 227 mg, about 228 mg, about 229 mg, about 230 mg, about 231 mg, about 232 mg, about 233 mg, about 234 mg, about 235 mg, about 236 mg, about 237 mg, about 238 mg, about 239 mg, about 240 mg, about 241 mg, about 242 mg, about 243 mg, about 244 mg, about 245 mg, about 246 mg, about 247 mg, about 248 mg, about 249 mg, about 250 mg, about 251 mg, about 252 mg, about 253 mg, about 254 mg, about 255 mg, about 256 mg, about 257 mg, about 258 mg, about 259 mg, about 260 mg, about 261 mg, about 262 mg, about 263 mg, about 264 mg, 271 mg, about 272 mg, about 273 mg, about 274 mg, about 275 mg, about 276 mg, about 277 mg, about 278 mg, about 279 mg, about 280 mg, about 281 mg, about 282 mg, about 283 mg, about 284 mg, about 285 mg, about 286 mg, about 287 mg, about 288 mg, about 289 mg, about 290 mg, about 291 mg, about 292 mg, about 293 mg, about 294 mg, about 295 mg, about 296 mg, about 297 mg, about 298 mg, about 299 mg, or about 300 mg, or more MDMA, or an equivalent dose of the pharmaceutically acceptable salt thereof.

In embodiments, the therapeutically effective amount of the enantiomerically enriched MDMA disclosed herein comprises a dose ranging from about 0.01 mg/kg to about 20 mg/kg MDMA, or an equivalent dose of the pharmaceutically acceptable salt thereof (e.g., about 1 mg/kg to about 15 mg/kg, about 5 mg/kg to about 15 mg/kg, about 10 mg/kg to about 20 mg/kg, about 3 mg/kg to about 15 mg/kg, or about 3 mg/kg to about 5 mg/kg MDMA, or an equivalent dose of the pharmaceutically acceptable salt thereof.) In embodiments, the therapeutically effective amount of enantiomerically enriched MDMA comprises a dose ranging from about 0.001 mg/kg to 50 mg/kg MDMA, or an equivalent dose of the pharmaceutically acceptable salt thereof. In embodiments, the therapeutically effective amount of enantiomerically enriched MDMA comprises a dose of about 0.001 mg/kg, about 0.01 mg/kg, about 0.1 mg/kg, 0.25 mg/kg, about 0.5 mg/kg, about 0.75 mg/kg, about 1.0 mg/kg, about 1.25 mg/kg, about 1.5 mg/kg, about 1.75 mg/kg, about 2.0 mg/mg, about 2.25 mg/kg, about 2.5 mg/kg, about 2.75 mg/kg, about 3.0 mg/kg, about 3.25 mg/kg, about 3.5 mg/kg, about 3.75 mg/kg, about 4.0 mg/kg, about 4.25 mg/kg, about 4.5 mg/kg, about 4.75 mg/kg, about 5.0 mg/kg, about 5.25 mg/kg, about 5.5 mg/kg, about 5.75 mg/kg, about 6.0 mg/kg, about 6.25 mg/kg, about 6.5 mg/kg, about 6.75 mg/kg, about 7.0 mg/kg, about 7.25 mg/kg, about 7.5 mg/kg, about 7.75 mg/kg, about 8.0 mg/kg, about 8.5 mg/kg, about 9.0 mg/kg, about 9.5 mg/kg, about 10.0 mg/kg, about 10.5 mg/kg, about 11.0 mg/kg, about 11.5 mg/kg, about 12.0 mg/kg, about 12.5 mg/kg, about 13.0 mg/kg, about 13.5 mg/kg, about 14.0 mg/kg, about 15.5 mg/kg, about 16.0 mg/kg, about 16.5 mg/kg, about 17.0 mg/kg, about 17.5 mg/kg, about 18.0 mg/kg, about 18.5 mg/kg, about 19.0 mg/kg, about 19.5 mg/kg, about 20.0 mg/kg, about 20.5 mg/kg, about 21 mg/kg, about 22, about mg/kg, about 23, about mg/kg, about 24 mg/kg, about 25 mg/kg, about 26 mg/kg, about 27 mg/kg, about 28 mg/kg, about 29 mg/kg, about 30 mg/kg, about 31 mg/kg, about 32, about mg/kg, about 33, about mg/kg, about 34 mg/kg, about 35 mg/kg, about 36 mg/kg, about 37 mg/kg, about 38 mg/kg, about 39 mg/kg, about 40 mg/kg, about 41 mg/kg, about 42, about mg/kg, about 43, about mg/kg, about 44 mg/kg, about 45 mg/kg, about 46 mg/kg, about 47 mg/kg, about 48 mg/kg, about 49 mg/kg, or about 50 mg/kg MDMA, or an equivalent dose of the pharmaceutically acceptable salt thereof.

In embodiments, the therapeutically effective amount of a CYP2D6 inhibitor comprises a dose in an amount effective to inhibit CYP2D6 metabolism of MDMA. In embodiments, the composition comprises a weight ratio of the inhibitor of CYP2D6 to R(−)-MDMA being about 1:1 or less than 1. For example, the composition comprises a weight ratio of the inhibitor of CYP2D6 to enantiomerically enriched MDMA ranging from about 1:1 to about 1:500 (e.g., about 1:2, about 1:5, about 1:10, about 1:20, about 1:30, about 1:40, about 1:50, about 1:60, about 1:70, about 1:80, about 1:90, about 1:100, about 1:110, about 1:120, about 1:30, about 1:140, about 1:150, about 1:160, about 1:170, about 1:180, about 1:190, about 1:200, about 1:210, about 1:220, about 1:230, about 1:240, about 1:250, about 1:260, about 1:270, about 1:280, about 1:290, about 1:300, about 1:310, about 1:320, about 1:330, about 1:340, about 1:350, about 1:360, about 1:370, about 1:380, about 1:390, about 1:400, about 1:410, about 1:420, about 1:430, about 1:440, about 1:450, about 1:460, about 1:470, about 1:480, about 1:490, or about 1:500, among other ratios within these enumerated ratios).

In embodiments, the therapeutically effective amount of a CYP2D6 inhibitor comprises a dose ranging from about 0.1 mg to about 750 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 5 1mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, about 120 mg, about 121 mg, about 122 mg, about 123 mg, about 124 mg, about 125 mg, about 126 mg, about 127 mg, about 128 mg, about 129 mg, about 130 mg, about 131 mg, about 132 mg, about 133 mg, about 134 mg, about 135 mg, about 136 mg, about 137 mg, about 138 mg, about 139 mg, about 140 mg, about 141 mg, about 142 mg, about 143 mg, about 144 mg, about 145 mg, about 146 mg, about 147 mg, about 148 mg, about 149 mg, about 150 mg, about 151 mg, about 152 mg, about 153 mg, about 154 mg, about 155 mg, about 156 mg, about 157 mg, about 158 mg, about 159 mg, about 160 mg, about 161 mg, about 162 mg, about 163 mg, about 164 mg, about 165 mg, about 166 mg, about 167 mg, about 168 mg, about 169 mg, about 170 mg, about 171 mg, about 172 mg, about 173 mg, about 174 mg, about 175 mg, about 176 mg, about 177 mg, about 178 mg, about 179 mg, about 180 mg, about 181 mg, about 182 mg, about 183 mg, about 184 mg, about 185 mg, about 186 mg, about 187 mg, about 188 mg, about 189 mg, about 190 mg, about 191 mg, about 192 mg, about 193 mg, about 194 mg, about 195 mg, about 196 mg, about 197 mg, about 198 mg, about 199 mg, about 200 mg, about 201 mg, about 202 mg, about 203 mg, about 204 mg, about 205 mg, about 206 mg, about 207 mg, about 208 mg, about 209 mg, about 210 mg, about 211 mg, about 212 mg, about 213 mg, about 214 mg, about 215 mg, about 216 mg, about 217 mg, about 218 mg, about 219 mg, about 220 mg, about 221 mg, about 222 mg, about 223 mg, about 224 mg, about 225 mg, about 226 mg, about 227 mg, about 228 mg, about 229 mg, about 230 mg, about 231 mg, about 232 mg, about 233 mg, about 234 mg, about 235 mg, about 236 mg, about 237 mg, about 238 mg, about 239 mg, about 240 mg, about 241 mg, about 242 mg, about 243 mg, about 244 mg, about 245 mg, about 246 mg, about 247 mg, about 248 mg, about 249 mg, about 250 mg, about 251 mg, about 252 mg, about 253 mg, about 254 mg, about 255 mg, about 256 mg, about 257 mg, about 258 mg, about 259 mg, about 260 mg, about 261 mg, about 262 mg, about 263 mg, about 264 mg, about 265 mg, about 266 mg, about 267 mg, about 268 mg, about 269 mg, about 270 mg, about 271 mg, about 272 mg, about 273 mg, about 274 mg, about 275 mg, about 276 mg, about 277 mg, about 278 mg, about 279 mg, about 280 mg, about 281 mg, about 282 mg, about 283 mg, about 284 mg, about 285 mg, about 286 mg, about 287 mg, about 288 mg, about 289 mg, about 290 mg, about 291 mg, about 292 mg, about 293 mg, about 294 mg, about 295 mg, about 296 mg, about 297 mg, about 298 mg, about 299 mg, or about 300 mg.

In embodiments, the therapeutically effective amount of a CYP2D6 inhibitor comprises a dose ranging from ranging from about 0.001 mg/kg to about 20 mg/kg (e.g., about 0.1 mg/kg to about 15 mg/kg, about 0.5 mg/kg to about 15 mg/kg, about 1 mg/kg to about 20 mg/kg, about 0.3 mg/kg to about 15 mg/kg, or about 0.3 mg/kg to about 5 mg/kg). In embodiments, the therapeutically effective amount of a CYP2D6 inhibitor comprises a dose ranging from about 0.0001 mg/kg to 50 mg/kg. In embodiments, the therapeutically effective amount of a CYP2D6 inhibitor comprises a dose of about 0.001 mg/kg, about 0.01 mg/kg, about 0.1 mg/kg, 0.25 mg/kg, about 0.5 mg/kg, about 0.75 mg/kg, about 1.0 mg/kg, about 1.25 mg/kg, about 1.5 mg/kg, about 1.75 mg/kg, about 2.0 mg/mg, about 2.25 mg/kg, about 2.5 mg/kg, about 2.75 mg/kg, about 3.0 mg/kg, about 3.25 mg/kg, about 3.5 mg/kg, about 3.75 mg/kg, about 4.0 mg/kg, about 4.25 mg/kg, about 4.5 mg/kg, about 4.75 mg/kg, about 5.0 mg/kg, about 5.25 mg/kg, about 5.5 mg/kg, about 5.75 mg/kg, about 6.0 mg/kg, about 6.25 mg/kg, about 6.5 mg/kg, about 6.75 mg/kg, about 7.0 mg/kg, about 7.25 mg/kg, about 7.5 mg/kg, about 7.75 mg/kg, about 8.0 mg/kg, about 8.5 mg/kg, about 9.0 mg/kg, about 9.5 mg/kg, about 10.0 mg/kg, about 10.5 mg/kg, about 11.0 mg/kg, about 11.5 mg/kg, about 12.0 mg/kg, about 12.5 mg/kg, about 13.0 mg/kg, about 13.5 mg/kg, about 14.0 mg/kg, about 15.5 mg/kg, about 16.0 mg/kg, about 16.5 mg/kg, about 17.0 mg/kg, about 17.5 mg/kg, about 18.0 mg/kg, about 18.5 mg/kg, about 19.0 mg/kg, about 19.5 mg/kg, about 20.0 mg/kg, about 20.5 mg/kg, about 21 mg/kg, about 22, about mg/kg, about 23, about mg/kg, about 24 mg/kg, about 25 mg/kg, about 26 mg/kg, about 27 mg/kg, about 28 mg/kg, about 29 mg/kg, about 30 mg/kg, about 31 mg/kg, about 32, about mg/kg, about 33, about mg/kg, about 34 mg/kg, about 35 mg/kg, about 36 mg/kg, about 37 mg/kg, about 38 mg/kg, about 39 mg/kg, about 40 mg/kg, about 41 mg/kg, about 42, about mg/kg, about 43, about mg/kg, about 44 mg/kg, about 45 mg/kg, about 46 mg/kg, about 47 mg/kg, about 48 mg/kg, about 49 mg/kg, or about 50 mg/kg.

In embodiments, the CYP2D6 inhibitor is administered before the first dose of enantiomerically enriched MDMA or a pharmaceutically acceptable salt thereof. In embodiments, the CYP2D6 inhibitor is administered as a single dose before the first dose of enantiomerically enriched MDMA or a pharmaceutically acceptable salt thereof. In embodiments, the CYP2D6 inhibitor is administered within about 0.5 hour, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours or about 15 hours of administration of the enantiomerically enriched MDMA or a pharmaceutically acceptable salt thereof. In embodiments, the CYP2D6 inhibitor is administered within about 12 hours of administration of the enantiomerically enriched MDMA or a pharmaceutically acceptable salt thereof. In embodiments, the CYP2D6 inhibitor is administered 1 to 10 days (e.g., 1 day, 2 days, 3 days, 4 days, 5 days, 6 days, 7 days, 8 days, 9 days, or 10 days) prior to administration of the enantiomerically enriched MDMA or a pharmaceutically acceptable salt thereof. In embodiments, the CYP2D6 inhibitor is administered 5 to 7 days prior to administration of the enantiomerically enriched MDMA or a pharmaceutically acceptable salt thereof. In embodiments, the CYP2D6 inhibitor is administered as a single daily dose for 1 day, 2 days, 3 days, 4 days, 5 days, 6 days, 7 days, 8 days, 9 days or 10 days before the first dose of enantiomerically enriched MDMA or a pharmaceutically acceptable salt thereof. In embodiments, the CYP2D6 inhibitor is co-administered (e.g., concomitantly or concurrently) with the enantiomerically enriched MDMA or a pharmaceutically acceptable salt thereof. In embodiments, the CYP2D6 inhibitor is administered before each dose of enantiomerically enriched MDMA or a pharmaceutically acceptable salt thereof. In embodiments, the CYP2D6 inhibitor is administered before a dose of enantiomerically enriched MDMA or a pharmaceutically acceptable salt thereof as needed.

In embodiments of kits comprising (i) a first composition comprsing racemic MDMA or MDMA comprising S(+)-MDMA in an enantiomeric excess relative to R(−)-MDMA or a pharmaceutically acceptable salt thereof; and (ii) a second composition comprising enantiomerically enriched MDMA comprising R(−)-MDMA in an enantiomeric excess relative to S(+)-MDMA or a pharmaceutically acceptable salt thereof, the effective amount of the first composition comprises a dose ranging from about 1 mg to about 1000 mg MDMA, or an equivalent dose of the pharmaceutically acceptable salt thereof, based on a 60 kg mammal subject (e.g., a human subject). In embodiments, the therapeutically effective amount of the first composition comprises a dose ranging from about 1 mg to about 750 mg MDMA, or an equivalent dose of the pharmaceutically acceptable salt thereof. In embodiments, the therapeutically effective amount of the first composition comprises a dose ranging from about 5 mg to about 500 mg, or about 10 mg to about 400 mg, or about 25 mg to about 300 mg, or about 75 mg to about 225 mg, or about 75 mg to about 850 mg, or about 250 mg to about 850 mg, or about 250 mg to about 350 mg MDMA, or an equivalent dose of the pharmaceutically acceptable salt thereof. In embodiments, the therapeutically effective amount comprises a dose ranging from about 25 mg to 50 mg MDMA, or an equivalent dose of the pharmaceutically acceptable salt thereof (e.g., about 20 mg, about 15 mg, about 10 mg, about 5 mg, about 1 mg, about 0.1 mg, about 0.01 mg, or about 0.001 mg). In embodiments, the therapeutically effective amount of the first composition comprises a dose of about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, about 120 mg, about 121 mg, about 122 mg, about 123 mg, about 124 mg, about 125 mg, about 126 mg, about 127 mg, about 128 mg, about 129 mg, about 130 mg, about 131 mg, about 132 mg, about 133 mg, about 134 mg, about 135 mg, about 136 mg, about 137 mg, about 138 mg, about 139 mg, about 140 mg, about 141 mg, about 142 mg, about 143 mg, about 144 mg, about 145 mg, about 146 mg, about 147 mg, about 148 mg, about 149 mg, about 150 mg, about 151 mg, about 152 mg, about 153 mg, about 154 mg, about 155 mg, about 156 mg, about 157 mg, about 158 mg, about 159 mg, about 160 mg, about 161 mg, about 162 mg, about 163 mg, about 164 mg, about 165 mg, about 166 mg, about 167 mg, about 168 mg, about 169 mg, about 170 mg, about 171 mg, about 172 mg, about 173 mg, about 174 mg, about 175 mg, about 176 mg, about 177 mg, about 178 mg, about 179 mg, about 180 mg, about 181 mg, about 182 mg, about 183 mg, about 184 mg, about 185 mg, about 186 mg, about 187 mg, about 188 mg, about 189 mg, about 190 mg, about 191 mg, about 192 mg, about 193 mg, about 194 mg, about 195 mg, about 196 mg, about 197 mg, about 198 mg, about 199 mg, about 200 mg, about 201 mg, about 202 mg, about 203 mg, about 204 mg, about 205 mg, about 206 mg, about 207 mg, about 208 mg, about 209 mg, about 210 mg, about 211 mg, about 212 mg, about 213 mg, about 214 mg, about 215 mg, about 216 mg, about 217 mg, about 218 mg, about 219 mg, about 220 mg, about 221 mg, about 222 mg, about 223 mg, about 224 mg, about 225 mg, about 226 mg, about 227 mg, about 228 mg, about 229 mg, about 230 mg, about 231 mg, about 232 mg, about 233 mg, about 234 mg, about 235 mg, about 236 mg, about 237 mg, about 238 mg, about 239 mg, about 240 mg, about 241 mg, about 242 mg, about 243 mg, about 244 mg, about 245 mg, about 246 mg, about 247 mg, about 248 mg, about 249 mg, about 250 mg, about 251 mg, about 252 mg, about 253 mg, about 254 mg, about 255 mg, about 256 mg, about 257 mg, about 258 mg, about 259 mg, about 260 mg, about 261 mg, about 262 mg, about 263 mg, about 264 mg, about 265 mg, about 266 mg, about 267 mg, about 268 mg, about 269 mg, about 270 mg, about 271 mg, about 272 mg, about 273 mg, about 274 mg, about 275 mg, about 276 mg, about 277 mg, about 278 mg, about 279 mg, about 280 mg, about 281 mg, about 282 mg, about 283 mg, about 284 mg, about 285 mg, about 286 mg, about 287 mg, about 288 mg, about 289 mg, about 290 mg, about 291 mg, about 292 mg, about 293 mg, about 294 mg, about 295 mg, about 296 mg, about 297 mg, about 298 mg, about 299 mg, or about 300 mg, or more MDMA, or an equivalent dose of the pharmaceutically acceptable salt thereof.

In embodiments, the therapeutically effective amount of the first compositions comprises a dose ranging from about 0.01 mg/kg to about 20 mg/kg MDMA, or an equivalent dose of the pharmaceutically acceptable salt thereof (e.g., about 1 mg/kg to about 15 mg/kg, about 5 mg/kg to about 15 mg/kg, about 10 mg/kg to about 20 mg/kg, about 3 mg/kg to about 15 mg/kg, or about 3 mg/kg to about 5 mg/kg MDMA, or an equivalent dose of the pharmaceutically acceptable salt thereof.) In embodiments, the therapeutically effective amount of the first composition comprises a dose ranging from about 0.001 mg/kg to 50 mg/kg MDMA, or an equivalent dose of the pharmaceutically acceptable salt thereof. In embodiments, the therapeutically effective amount of the first composition comprises a dose of about 0.001 mg/kg, about 0.01 mg/kg, about 0.1 mg/kg, 0.25 mg/kg, about 0.5 mg/kg, about 0.75 mg/kg, about 1.0 mg/kg, about 1.25 mg/kg, about 1.5 mg/kg, about 1.75 mg/kg, about 2.0 mg/mg, about 2.25 mg/kg, about 2.5 mg/kg, about 2.75 mg/kg, about 3.0 mg/kg, about 3.25 mg/kg, about 3.5 mg/kg, about 3.75 mg/kg, about 4.0 mg/kg, about 4.25 mg/kg, about 4.5 mg/kg, about 4.75 mg/kg, about 5.0 mg/kg, about 5.25 mg/kg, about 5.5 mg/kg, about 5.75 mg/kg, about 6.0 mg/kg, about 6.25 mg/kg, about 6.5 mg/kg, about 6.75 mg/kg, about 7.0 mg/kg, about 7.25 mg/kg, about 7.5 mg/kg, about 7.75 mg/kg, about 8.0 mg/kg, about 8.5 mg/kg, about 9.0 mg/kg, about 9.5 mg/kg, about 10.0 mg/kg, about 10.5 mg/kg, about 11.0 mg/kg, about 11.5 mg/kg, about 12.0 mg/kg, about 12.5 mg/kg, about 13.0 mg/kg, about 13.5 mg/kg, about 14.0 mg/kg, about 15.5 mg/kg, about 16.0 mg/kg, about 16.5 mg/kg, about 17.0 mg/kg, about 17.5 mg/kg, about 18.0 mg/kg, about 18.5 mg/kg, about 19.0 mg/kg, about 19.5 mg/kg, about 20.0 mg/kg, about 20.5 mg/kg, about 21 mg/kg, about 22, about mg/kg, about 23, about mg/kg, about 24 mg/kg, about 25 mg/kg, about 26 mg/kg, about 27 mg/kg, about 28 mg/kg, about 29 mg/kg, about 30 mg/kg, about 31 mg/kg, about 32, about mg/kg, about 33, about mg/kg, about 34 mg/kg, about 35 mg/kg, about 36 mg/kg, about 37 mg/kg, about 38 mg/kg, about 39 mg/kg, about 40 mg/kg, about 41 mg/kg, about 42, about mg/kg, about 43, about mg/kg, about 44 mg/kg, about 45 mg/kg, about 46 mg/kg, about 47 mg/kg, about 48 mg/kg, about 49 mg/kg, or about 50 mg/kg MDMA, or an equivalent dose of the pharmaceutically acceptable salt thereof.

In embodiments, the effective amount of the second composition comprises a dose ranging from about 1 mg to about 1000 mg MDMA, or an equivalent dose of the pharmaceutically acceptable salt thereof, based on a 60 kg mammal subject (e.g., a human subject). In embodiments, the therapeutically effective amount comprises a dose ranging from about 1 mg to about 750 mg MDMA, or an equivalent dose of the pharmaceutically acceptable salt thereof. In embodiments, the therapeutically effective amount comprises a dose ranging from about 5 mg to about 500 mg, or about 10 mg to about 400 mg, or about 25 mg to about 300 mg, or about 75 mg to about 225 mg, or about 75 mg to about 850 mg, or about 250 mg to about 850 mg, or about 250 mg to about 350 mg MDMA, or an equivalent dose of the pharmaceutically acceptable salt thereof. In embodiments, the therapeutically effective amount comprises a dose ranging from about 25 mg to 50 mg MDMA, or an equivalent dose of the pharmaceutically acceptable salt thereof (e.g., about 20 mg, about 15 mg, about 10 mg, about 5 mg, about 1 mg, about 0.1 mg, about 0.01 mg, or about 0.001 mg). In embodiments, the therapeutically effective amount comprises a dose of about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, about 120 mg, about 121 mg, about 122 mg, about 123 mg, about 124 mg, about 125 mg, about 126 mg, about 127 mg, about 128 mg, about 129 mg, about 130 mg, about 131 mg, about 132 mg, about 133 mg, about 134 mg, about 135 mg, about 136 mg, about 137 mg, about 138 mg, about 139 mg, about 140 mg, about 141 mg, about 142 mg, about 143 mg, about 144 mg, about 145 mg, about 146 mg, about 147 mg, about 148 mg, about 149 mg, about 150 mg, about 151 mg, about 152 mg, about 153 mg, about 154 mg, about 155 mg, about 156 mg, about 157 mg, about 158 mg, about 159 mg, about 160 mg, about 161 mg, about 162 mg, about 163 mg, about 164 mg, about 165 mg, about 166 mg, about 167 mg, about 168 mg, about 169 mg, about 170 mg, about 171 mg, about 172 mg, about 173 mg, about 174 mg, about 175 mg, about 176 mg, about 177 mg, about 178 mg, about 179 mg, about 180 mg, about 181 mg, about 182 mg, about 183 mg, about 184 mg, about 185 mg, about 186 mg, about 187 mg, about 188 mg, about 189 mg, about 190 mg, about 191 mg, about 192 mg, about 193 mg, about 194 mg, about 195 mg, about 196 mg, about 197 mg, about 198 mg, about 199 mg, about 200 mg, about 201 mg, about 202 mg, about 203 mg, about 204 mg, about 205 mg, about 206 mg, about 207 mg, about 208 mg, about 209 mg, about 210 mg, about 211 mg, about 212 mg, about 213 mg, about 214 mg, about 215 mg, about 216 mg, about 217 mg, about 218 mg, about 219 mg, about 220 mg, about 221 mg, about 222 mg, about 223 mg, about 224 mg, about 225 mg, about 226 mg, about 227 mg, about 228 mg, about 229 mg, about 230 mg, about 231 mg, about 232 mg, about 233 mg, about 234 mg, about 235 mg, about 236 mg, about 237 mg, about 238 mg, about 239 mg, about 240 mg, about 241 mg, about 242 mg, about 243 mg, about 244 mg, about 245 mg, about 246 mg, about 247 mg, about 248 mg, about 249 mg, about 250 mg, about 251 mg, about 252 mg, about 253 mg, about 254 mg, about 255 mg, about 256 mg, about 257 mg, about 258 mg, about 259 mg, about 260 mg, about 261 mg, about 262 mg, about 263 mg, about 264 mg, 271 mg, about 272 mg, about 273 mg, about 274 mg, about 275 mg, about 276 mg, about 277 mg, about 278 mg, about 279 mg, about 280 mg, about 281 mg, about 282 mg, about 283 mg, about 284 mg, about 285 mg, about 286 mg, about 287 mg, about 288 mg, about 289 mg, about 290 mg, about 291 mg, about 292 mg, about 293 mg, about 294 mg, about 295 mg, about 296 mg, about 297 mg, about 298 mg, about 299 mg, or about 300 mg, or more MDMA, or an equivalent dose of the pharmaceutically acceptable salt thereof.

In embodiments, the therapeutically effective amount of the second compositions comprises a dose ranging from about 0.01 mg/kg to about 20 mg/kg MDMA, or an equivalent dose of the pharmaceutically acceptable salt thereof (e.g., about 1 mg/kg to about 15 mg/kg, about 5 mg/kg to about 15 mg/kg, about 10 mg/kg to about 20 mg/kg, about 3 mg/kg to about 15 mg/kg, or about 3 mg/kg to about 5 mg/kg MDMA, or an equivalent dose of the pharmaceutically acceptable salt thereof.) In embodiments, the therapeutically effective amount of the second composition comprises a dose ranging from about 0.001 mg/kg to 50 mg/kg MDMA, or an equivalent dose of the pharmaceutically acceptable salt thereof. In embodiments, the therapeutically effective amount of the second composition comprises a dose of about 0.001mg/kg, about 0.01 mg/kg, about 0.1 mg/kg, 0.25 mg/kg, about 0.5 mg/kg, about 0.75 mg/kg, about 1.0 mg/kg, about 1.25 mg/kg, about 1.5 mg/kg, about 1.75 mg/kg, about 2.0 mg/mg, about 2.25 mg/kg, about 2.5 mg/kg, about 2.75 mg/kg, about 3.0 mg/kg, about 3.25 mg/kg, about 3.5 mg/kg, about 3.75 mg/kg, about 4.0 mg/kg, about 4.25 mg/kg, about 4.5 mg/kg, about 4.75 mg/kg, about 5.0 mg/kg, about 5.25 mg/kg, about 5.5 mg/kg, about 5.75 mg/kg, about 6.0 mg/kg, about 6.25 mg/kg, about 6.5 mg/kg, about 6.75 mg/kg, about 7.0 mg/kg, about 7.25 mg/kg, about 7.5 mg/kg, about 7.75 mg/kg, about 8.0 mg/kg, about 8.5 mg/kg, about 9.0 mg/kg, about 9.5 mg/kg, about 10.0 mg/kg, about 10.5 mg/kg, about 11.0 mg/kg, about 11.5 mg/kg, about 12.0 mg/kg, about 12.5 mg/kg, about 13.0 mg/kg, about 13.5 mg/kg, about 14.0 mg/kg, about 15.5 mg/kg, about 16.0 mg/kg, about 16.5 mg/kg, about 17.0 mg/kg, about 17.5 mg/kg, about 18.0 mg/kg, about 18.5 mg/kg, about 19.0 mg/kg, about 19.5 mg/kg, about 20.0 mg/kg, about 20.5 mg/kg, about 21 mg/kg, about 22, about mg/kg, about 23, about mg/kg, about 24 mg/kg, about 25 mg/kg, about 26 mg/kg, about 27 mg/kg, about 28 mg/kg, about 29 mg/kg, about 30 mg/kg, about 31 mg/kg, about 32, about mg/kg, about 33, about mg/kg, about 34 mg/kg, about 35 mg/kg, about 36 mg/kg, about 37 mg/kg, about 38 mg/kg, about 39 mg/kg, about 40 mg/kg, about 41 mg/kg, about 42, about mg/kg, about 43, about mg/kg, about 44 mg/kg, about 45 mg/kg, about 46 mg/kg, about 47 mg/kg, about 48 mg/kg, about 49 mg/kg, or about 50 mg/kg MDMA, or an equivalent dose of the pharmaceutically acceptable salt thereof.

In embodiments, the first composition comprising racemic MDMA or MDMA comprising S(+)-MDMA in an enantiomeric excess relative to R(−)-MDMA is administered before the first dose of the second composition comprising enantiomerically enriched MDMA comprising R(−)-MDMA in an enantiomeric excess relative to S(+)-MDMA. In embodiments, the first composition is administered as a single dose before the first dose of the second composition. In embodiments, the first composition is administered within about 0.5 hour, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours or about 15 hours of administration of the second composition. In embodiments, the first composition is administered within about 12 hours of the second composition. In embodiments, the first composition is administered 1 to 10 days (e.g., 1 day, 2 days, 3 days, 4 days, 5 days, 6 days, 7 days, 8 days, 9 days, or 10 days) prior to administration of the second pharmaceutical composition. In embodiments, the first composition is administered 5 to 7 days prior to administration of the second composition. In embodiments, the first composition is administered as a single daily dose for 1 day, 2 days, 3 days, 4 days, 5 days, 6 days, 7 days, 8 days, 9 days or 10 days before the first dose of the second composition. In embodiments, the first composition is co-administered (e.g., concomitantly or concurrently) with the second composition. In embodiments, the first composition is administered before each dose of the second composition. In embodiments, the first composition is administered before a dose of the second composition as needed.

The effective amounts may be provided as a single dose or on regular schedule, i.e., on a daily, weekly, monthly, or yearly basis or an irregular schedule with varying administration days, weeks, months, etc. To reduce the occurrence of possible side effect associated with the dose, an effective amount may be provided as a split dose where the single dose is split into two doses, that are administered apart, usually over several hours.

The effective amounts may be provided as a single dose or on regular schedule, i.e., on a daily, weekly, monthly, or yearly basis or on an irregular schedule with varying administration days, weeks, months, etc. To reduce the occurrence of possible side effect associated with the dose, an effective amount may be provided as a split dose, where the single dose is split into two doses, that are administered apart, usually over several hours. For example, a single dose may be split into two doses, administered 1 hour apart, 2 hours apart, 3 hours apart, 4 hours apart, 5 hours apart, 6 hours apart, 7 hours apart, 8 hours apart, or more. Alternatively, the therapeutically effective amount to be administered may vary. In aspects, the therapeutically effective amount for the first dose is higher than the therapeutically effective amount for one or more of the subsequent doses. In aspects, the therapeutically effective amount for the first dose is lower than the therapeutically effective amount for one or more of the subsequent doses. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every 2 weeks, about every 3 weeks, about every month, about every 2 months, about every 3 months and about every 6 months. The number and frequency of dosages corresponding to a completed course of therapy will be determined according to the judgment of a health-care practitioner.

In embodiments, the disclosure provides compositions, kits and dosage regimens that provide an effective serum or plasma concentration of the active agent in the subject. As used herein, an “effective concentration” is characterized by the concentration of agent (e.g., R(−)-MDMA, or a metabolite thereof such as, for example, R(−)-MDA, or a combination thereof) in the plasma or serum that is effective to exert a desired or intended physiological response. In embodiments, the compositions, kits and dosage regimens provide a plasma concentration of R(−)-MDMA from about 150 to about 1000 ng/mL, about 200 to about 1000 ng/ml, about 250 to about 1000 ng/ml, about 300 to about 1000 ng/mL, about 350 to about 1000 ng/ml, about 400 to about 1000 ng/mL, about 450 to about 1000 ng/ml, about 500 to about 1000 ng/ml, about 550 to about 1000 ng/mL, about 600 to about 1000 ng/mL, about 650 to about 1000 ng/ml, about 700 to about 1000 ng/mL, about 750 to about 1000 ng/ml, or about 800 to about 1000 ng/ml, inclusive of any sub-range within those plasma concentration values. In embodiments, the plasma concentration of R(−)-MDMA achieved can range from about 200 to about 800 ng/ml, about 250to about 800 ng/ml, about 300 to about 800 ng/ml, about 350 to about 800 ng/ml, about 400 to about 800 ng/mL, about 450 to about 800 ng/ml, about 500 to about 800 ng/mL, about 550 to about 800 ng/mL, about 600 to about 800 ng/mL, about 650 to about 800 ng/ml, or about 700 to about 800 ng/mL. In embodiments, the compositions, kits and dosage regimens provide a plasma concentration of R(−)-MDMA of about 225, 250, 275, 300, 325, 350, 375, 400, 425, 450, 475, 500, 525, 550, 575, 600, 625 650, 675, 700, 725, 750, 775, 800, 825, 850, 875, 900, 925, 950, 975, to about 1000 ng/mL.

In embodiments, the compositions, kits and dosage regimens provide a total plasma concentration of R(−)-MDA from about 150 to about 1000 ng/mL, about 200 to about 1000 ng/ml, about 250 to about 1000 ng/mL, about 300 to about 1000 ng/mL, about 350 to about 1000 ng/ml, about 400 to about 1000 ng/ml, about 450 to about 1000 ng/ml, about 500 to about 1000 ng/ml, about 550 to about 1000 ng/mL, about 600 to about 1000 ng/mL, about 650 to about 1000 ng/ml, about 700 to about 1000 ng/mL, about 750 to about 1000 ng/ml, or about 800 to about 1000 ng/ml, inclusive of any sub-range within those plasma concentration values. In embodiments, the plasma concentration of R(−)-MDA achieved can range from about 200 to about 800 ng/ml, about 250 to about 800 ng/mL, about 300 to about 800 ng/ml, about 350 to about 800 ng/ml, about 400 to about 800 ng/mL, about 450 to about 800 ng/mL, about 500 to about 800 ng/ml, about 550 to about 800 ng/mL, about 600 to about 800 ng/ml, about 650 to about 800 ng/ml, or about 700 to about 800 ng/mL. In embodiments, the compositions, kits and dosage regimens provide a plasma concentration of R(−)-MDA of about 225, 250, 275, 300, 325, 350, 375, 400, 425, 450, 475, 500, 525, 550, 575, 600, 625 650, 675, 700, 725, 750, 775, 800, 825, 850, 875, 900, 925, 950, 975, to about 1000 ng/mL.

In yet some further embodiments, the compositions, kits and dosage regimens provide a total plasma concentration of combined R(−)-MDMA and R(−)-MDA from about 150 to about 1000 ng/mL, about 200 to about 1000 ng/mL, about 250 to about 1000 ng/ml, about 300 to about 1000 ng/mL, about 350 to about 1000 ng/mL, about 400 to about 1000 ng/ml, about 450 to about 1000 ng/mL, about 500 to about 1000 ng/mL, about 550 to about 1000 ng/mL, about 600 to about 1000 ng/ml, about 650 to about 1000 ng/ml, about 700 to about 1000 ng/ml, about 750 to about 1000 ng/mL, or about 800 to about 1000 ng/mL, inclusive of any sub-range within those plasma concentration values. In embodiments, the total plasma concentration of combined R(−)-MDMA and R(−)-MDA achieved can range from about 200 to about 800 ng/ml, about 250 to about 800 ng/mL, about 300 to about 800 ng/mL, about 350 to about 800 ng/ml, about 400 to about 800 ng/mL, about 450 to about 800 ng/ml, about 500 to about 800 ng/ml, about 550 to about 800 ng/mL, about 600 to about 800 ng/mL, about 650 to about 800 ng/mL, or about 700 to about 800 ng/mL. In embodiments, the compositions, kits and dosage regimens provide a total plasma concentration of combined R(−)-MDMA and R(−)-MDA of about 225, 250, 275, 300, 325, 350, 375, 400, 425, 450, 475, 500, 525, 550, 575, 600, 625 650, 675, 700, 725, 750, 775, 800, 825, 850, 875, 900, 925, 950, 975, to about 1000 ng/mL.

### EXAMPLES

The following examples are provided to further illustrate the embodiments of the present disclosure but are not intended to limit the scope of the claims. While they are typical of those that might be used, other procedures, methodologies, or techniques known to those skilled in the art may alternatively be used.

### Example 1. (R)(−)-MDMA Phase 1 Clinical Study

A Phase 1 clinical study was conducted to evaluate the effects of orally administered R(−)-MDMA in healthy volunteers. A total of 32 healthy participants (mean age of 28.5 years (ranging from 19-47 years); 58.3% female, 41.7% male) enrolled in a four-cohort, single-ascending dose, randomized, double-blind, placebo-controlled study. The cohorts (all fasted, with the exception of one arm) received 75 mg, 125 mg, 175 mg or 225 mg of R(−)-MDMA or placebo in a 6+2 design.

Enantiomerically enriched R(−)-MDMA (98.8% R(−)-MDMA HCl by achiral HPLC; 100.0% R(−)-MDMA HCl by chiral chromatography) was prepared as powder (API) in capsules (HPMC capsule shells) at two dosages, 29.7 mg drug substance (corresponding to 25 mg of R(−)-MDMA free base) and 118.9 mg drug substance (corresponding to 100 mg of R(−)-MDMA free base). The primary endpoints assessed the safety, tolerability, and pharmacokinetics (PK), as well as exploratory pharmacodynamic (PD) observations, as discussed below. Treatment-related adverse events (AEs) were in line with expectations (e.g., nausea at 29.2%, headache at 16.7%) and generally dose dependent. There were no study discontinuations, and no serious or severe AEs. Non-clinically significant increases in blood pressure and heart rate were observed, though such changes showed limited dose dependency. Peak body temperatures fell within the normal range. Bruxism was observed in a single subject at the highest dose (225 mg).

### Pharmacokinetics (PK)

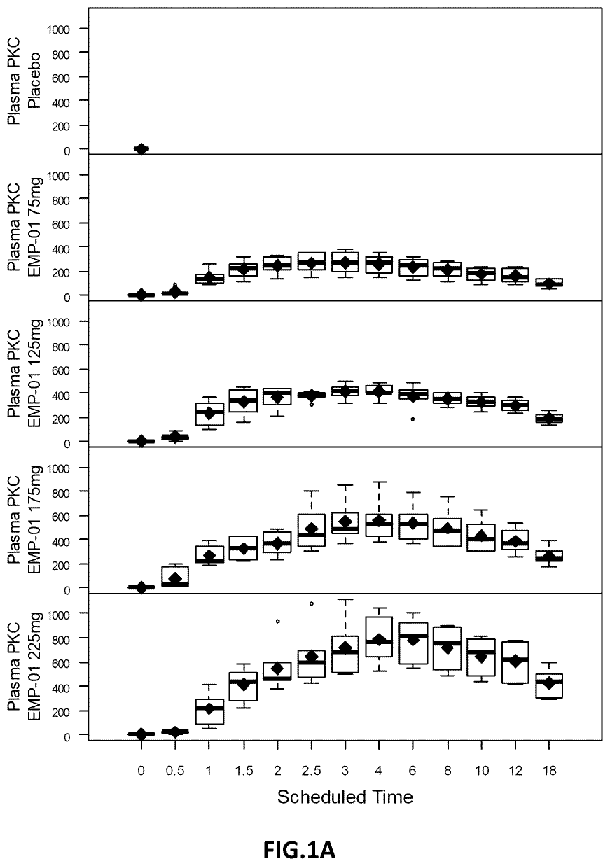

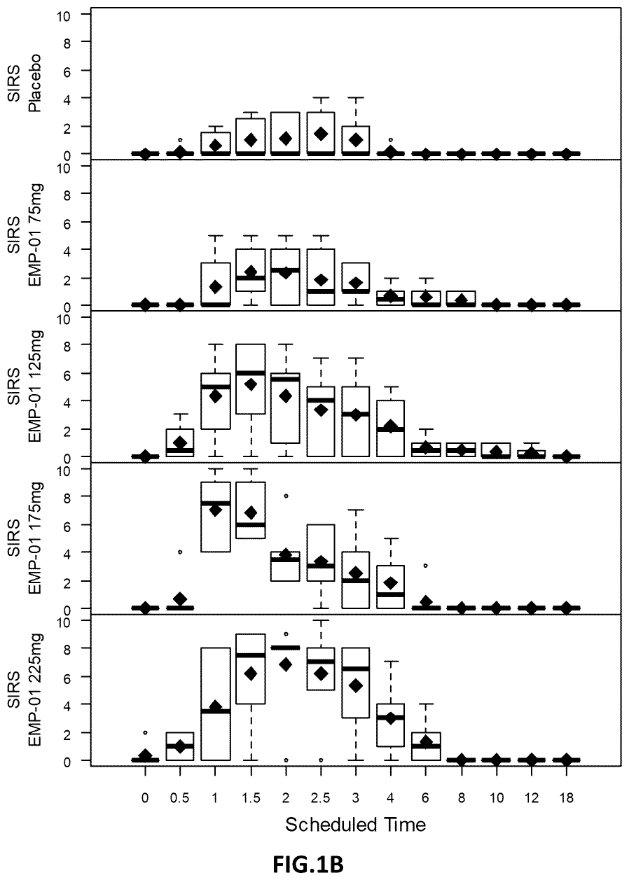

The data demonstrates that R(−)-MDMA was well-tolerated with dose-dependent pharmacokinetics. No patterns in the data suggested any substantial PK food effects. Illustrative plasma PK concentrations, in ng/mL, for each cohort (placebo, 75 mg, 125 mg, 175 mg, 225 mg) are presented in FIG. 1B as a function of time.

Systemic exposure (e.g., Cmax and AUC) results exhibited an increase with increasing doses in a generally dose-dependent manner, in-line with expectations based on prior reports for racemic MDMA. R(−)-MDMA is known to be metabolized more slowly than S(+)-MDMA; thus, the observation of a substantially higher AUC₀₋₂₄ₕ with R(−)-MDMA following dosing in this study as compared to prior reports for AUC₀₋₂₄ₕ for total MDMA following racemic-MDMA dosing may attribute to the content of R(−)-MDMA in the dosage form that was administered.

At higher doses, it was observed that systemic exposure (Cmax/AUC) to the active metabolite, R(−)-MDA, increased with doses in a slightly less than dose-dependent manner, which suggests that the rate of R(−)-MDMA metabolism (i.e., production of R(−)-MDA) at the higher doses is slower than at lower dosages.

### Exploratory Pharmacodynamics (PD)

### Spoken Intensity Rating Scale (SIRS) Result

The SIRS results showed a very strong dose response (FIG. A) and indicated that the peak SIRS occurred shortly after detectable increases in plasma PK concentration, but much earlier than the plasma PK Tmax at each dose (FIG. 1B).

### Visual Analog Scale (VAS) Results

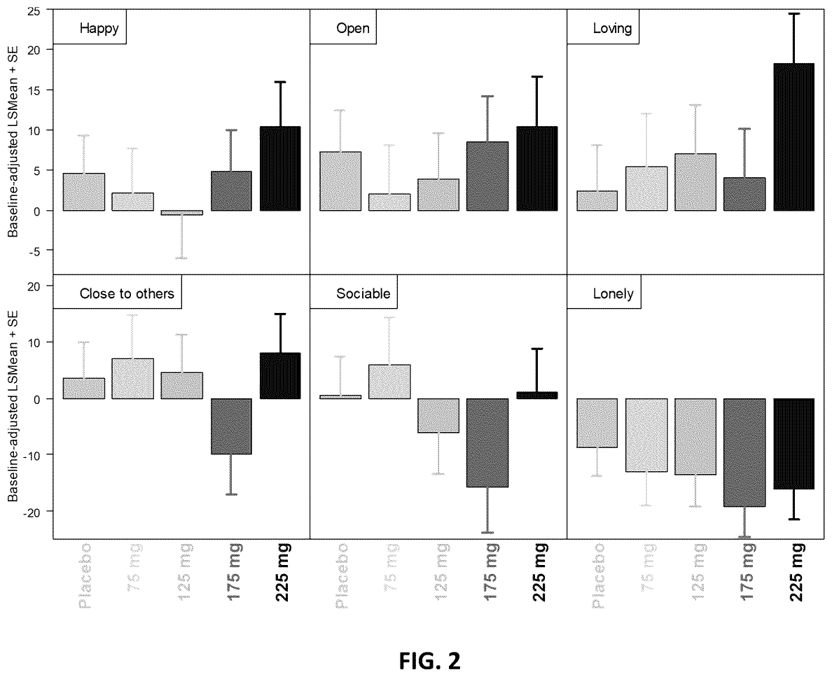

Visual Analogue Scales (VAS) was used to measure subjective experience, including experiences such as Happy, Open, Loving, Close to Others, Sociable, and Lonely. As shown in FIG. 2, the VAS scores for Happy, Open, Loving, and Lonly showed changes from baseline (prior to treatment) in a dose-dependent manner compared to placebo control while the VAS scores for Close to Others and Sociable showed no changes from baseline compared to placebo in all cohorts. Some signal increases were observable for acute increases in the VAS scores of Happy, Open, and Loving experiences, and an acute decrease associated with Lonely (FIG. 2). However, the degree of those signals was far less than previously reported signals for racemic MDMA. These effects for R(−)-MDMA were observable within the acute treatment phase and were dimished (relative to baseline) after 18 hours post-dosing. These results stand in contrast to other studies of racemic MDMA in healthy human subjects, where acute and substantial increases have been observed for Happy, Open, Loving, Close to Others, and Sociable. However, R(−)-MDMA did show a consistent pattern in the 225 mg cohort, with highest changes relative to baseline and with similar directionality as the effects reported for racemic MDMA.

### Facial Emotion Recognition Test (FERT)

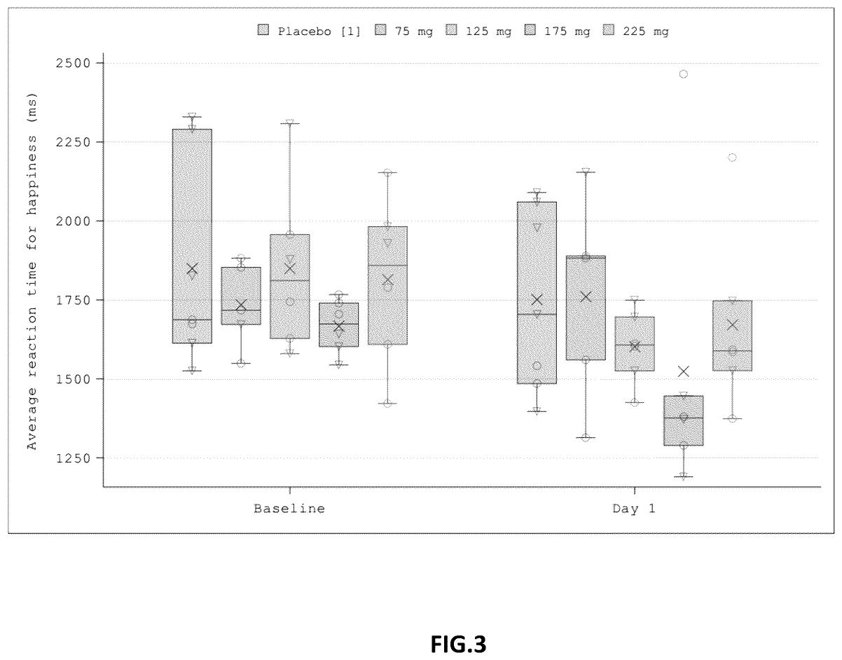

There was a reduction in the mean happiness reaction time in the cohorts receiving 125 mg, 175 mg, and 225 mg of R(−)-MDMA where happiness was correctly identified, evidenced through larger change from baseline scores, in comparison to placebo (FIG. 3). In addition, there was a greater reduction in the median reaction time after treatment with 125 mg and 175 mg, evidenced through greater change from baseline scores, in comparison to placebo (FIG. 3).

### Multifaceted Empathy Test (MET)

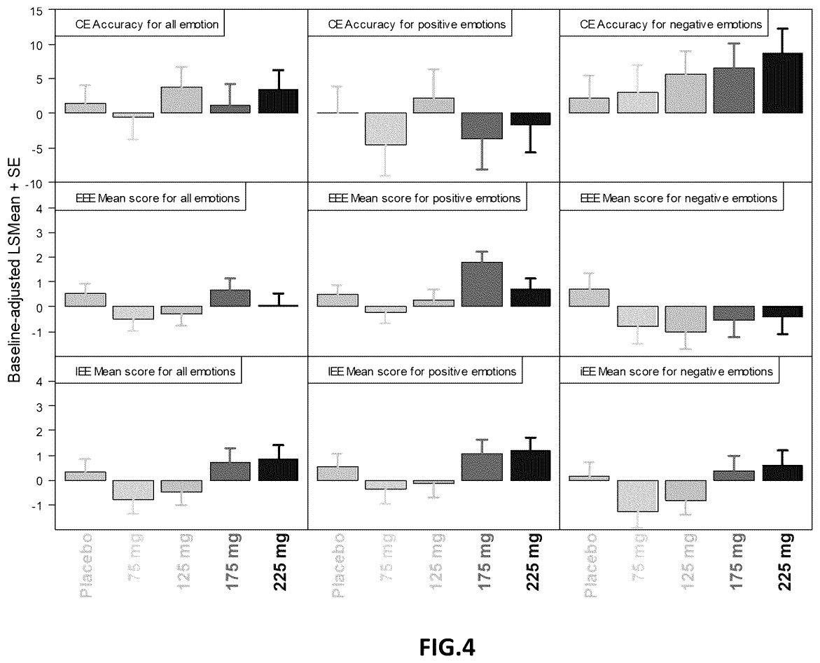

The Multifaceted Empathy Test (MET) measures cognitive empathy (CE), explicit emotional empathy (EEE) and implicit emotional empathy (IEE). Hurlemann R, et al., (2010) Oxytocin enhances amygdala-dependent, socially reinforced learning and emotional empathy in humans. J Neurosci 30:4999-5007. The MET LSmean scores for CE all emotions, CE negative emotions, EEE positive emotions, IEE all emotions, IEE positive emotions and IEE negative emotions showed changes from baseline in a dose-dependent manner (FIG. 4). The MET scores for CE positive emotions, EEE all emotions and EEE negative emotions showed no changes from baseline compared to placebo in all cohorts (FIG. 4).

### Neuroendocrine Effects

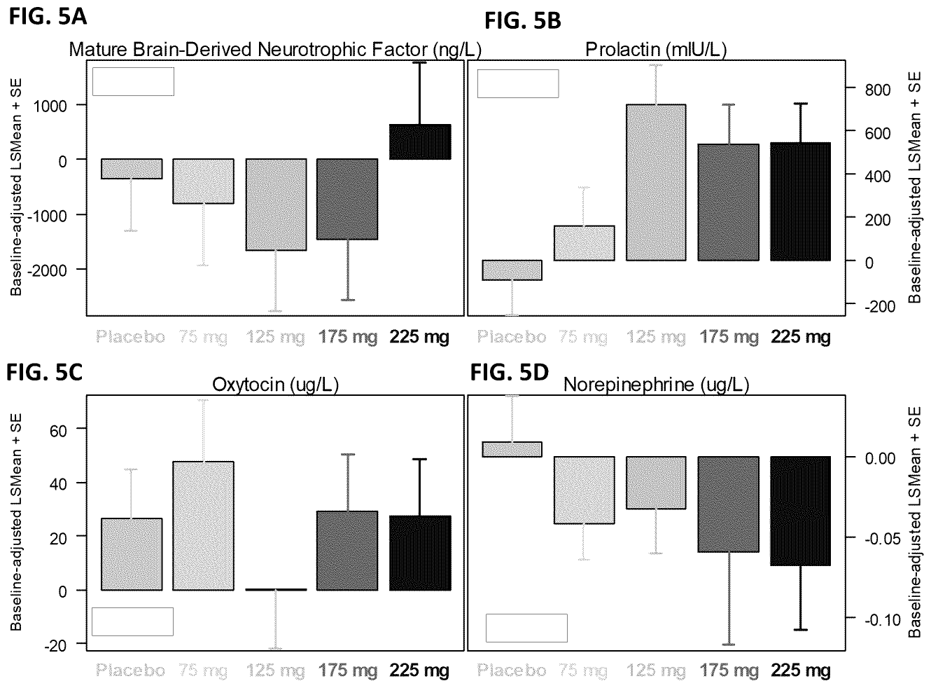

Among the hormones measured among the cohorts, a consistent increase in the concentration of prolactin was observed in response to R(−)-MDMA doses, compared with the placebo group (FIG. 5B). This suggests involvement of serotonergic signaling, as increases in prolactin is mediated by 5-HT release.

In contrast, no obvious change from baseline was observed in the concentration of oxytocin (FIG. 5C) or mature brain-derived neurotrophic factor (FIG. 5A) in any of the cohorts when compared with the placebo group. As illustrated in FIG. 5C, none of the R(−)-MDMA cohorts exhibited an oxytocin increase above the placebo group. This result stands in contrast with prior studies of racemic MDMA that suggest racemic MDMA can stimulate a strong (e.g., 5-6x) oxytocin release through serotonergic signaling (see, e.g., Hysek, C. M., et al., Psychopharmacology (Berl). 2012 July;222 (2): 293-302; Hysek, C. M., et al., Soc Cogn Affect Neurosci., 2014 November;9(11): 1645-52; and Thompson, M. R., et al., Neuroscience (2007 May 11); 146(2): 509-14).

### 5-Dimensions-Altered States of Consciousness (5D-ASC)

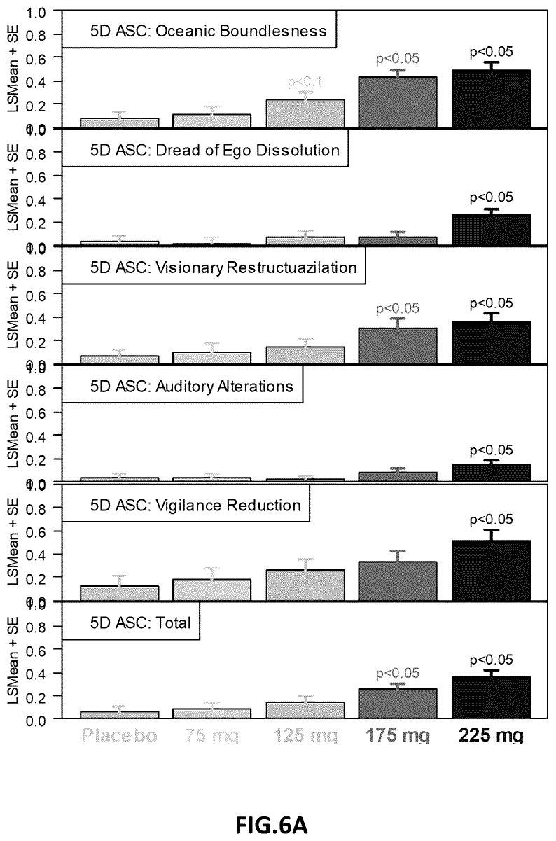

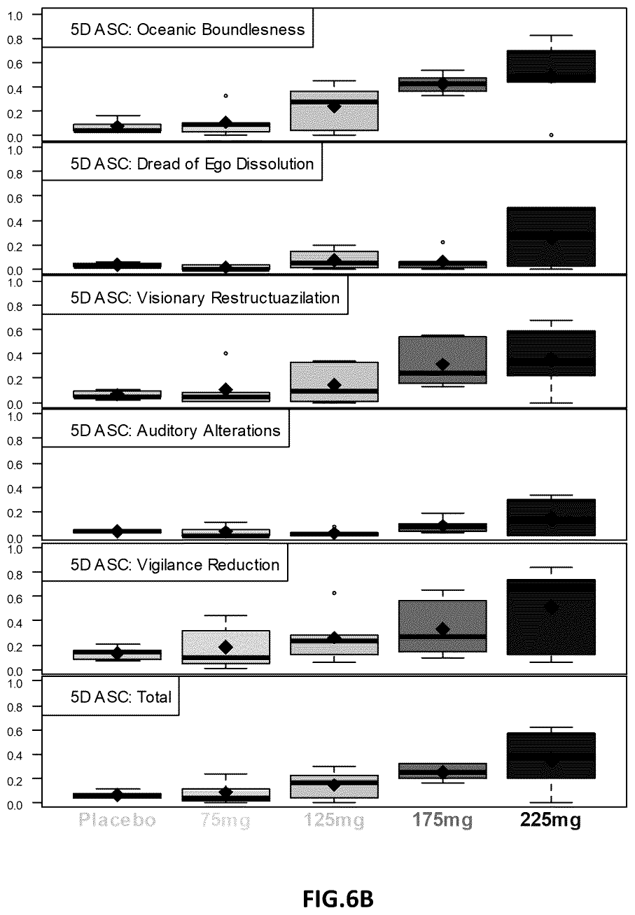

As shown in FIGS. 6A-6B, subjects reported a dose-dependent increase in all measured subcomponents (e.g., Oceanic Boundlessness, Dread of Ego Dissolution, Visionary Restructuralization, Auditory Alterations, and Vigilance Reduction) and the total score, with statistical significance at higher doses. The results of the 5D-ASC assessment suggest that R(−)-MDMA possesses a profile different from that of racemic MDMA, and has more psychedelic-like characteristics. This is generally unexpected given the prior results that have been reported for MDMA. One notable finding from the assessment is the ratio of Oceanic Boundlessness (OB) to Dread of Ego Dissolution (DED), which are considered a useful metric. OB is positively correlated with psychedelic-mediated antidepressant efficacy while DED is negatively correlated. Over the dosage range, a very favorable ratio was observed in the 175 mg cohort. The OB-DED ratio is consistent with the 11-Dimensional Altered States of Consciousness (11D-ASC, see below).

### 11-Dimensions-Altered States of Consciousness (11D-ASC)

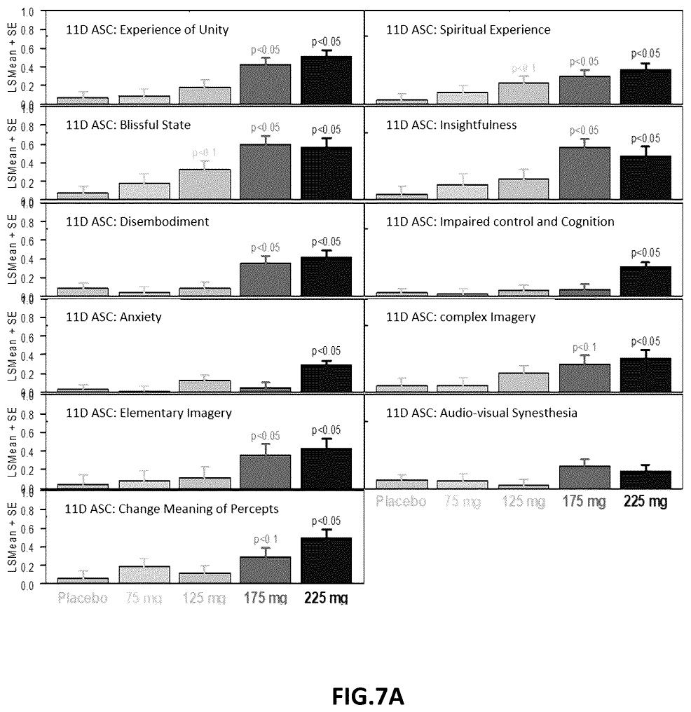

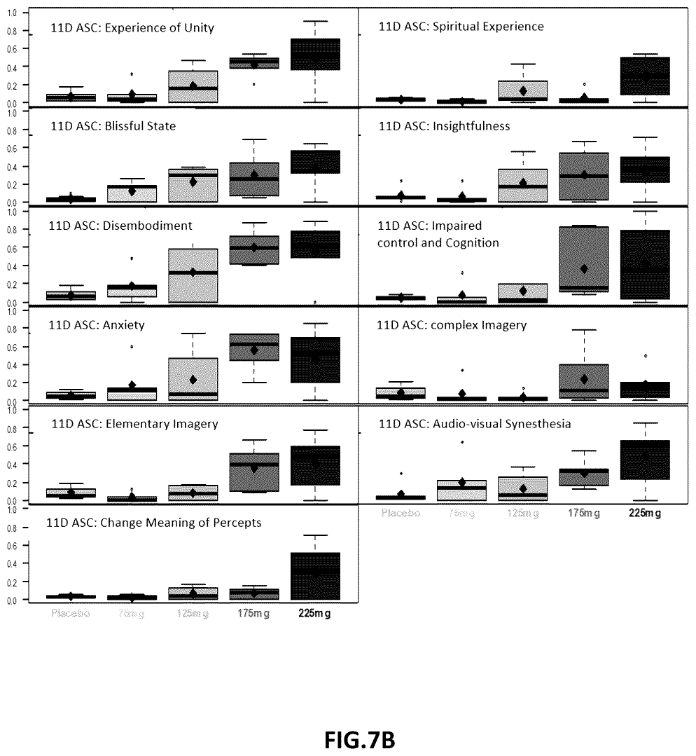

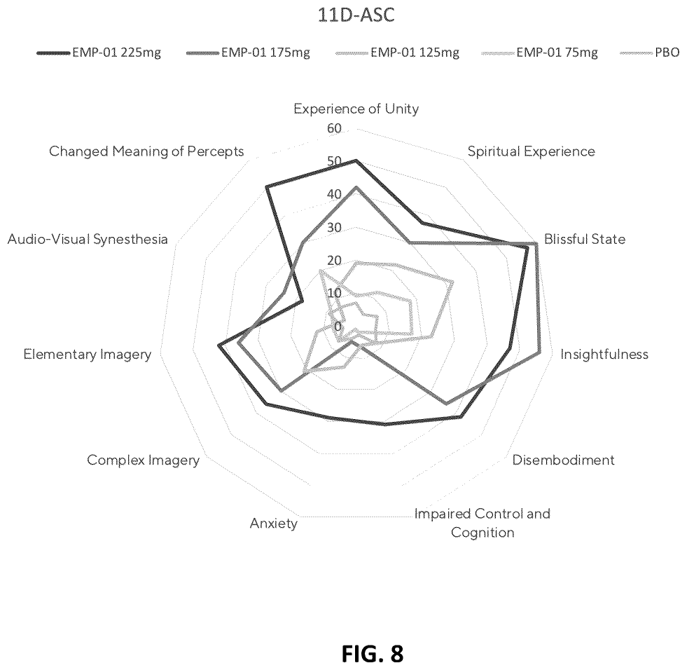

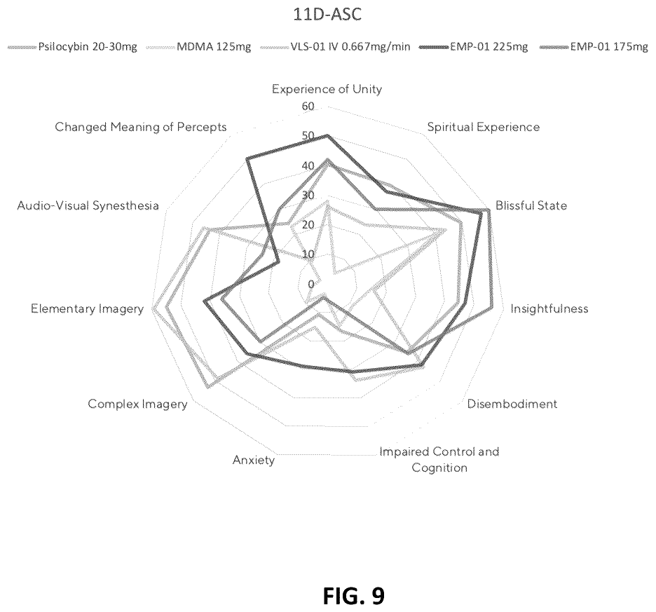

The results of the 11D-ASC assessment further confirms the unexpected psychedelic-like response associated with R(−)-MDMA. As shown in FIGS. 7A-7B, the subjects reported a dose-dependent increase in all measured subcomponents (e.g., Experience of Unity, Blissful State, Disembodiment, Anxiety, Elementary Imagery, Change Meaning of Percepts, Spiritual Experience, Insightfulness, Impaired Control and Cognition, Complex Imagery, and Audio-visual Synesthesia) and the total score, with statistical significance at higher doses. FIG. 8 plots the effects of R(−)-MDMA at 75 mg, 125 mg, 175 mg, and 225 mg on both simple and complex imagery, perceptual alterations, and synesthesia. FIG. 9 shows the comparison of psilocybin, intravenous dimethyltryptamine (IV VLS-01), racemic MDMA, and R(−)-MDMA (EMP-01) at 175 mg and 225 mg doses. The response profile of R(−)-MDMA has many similarities to that of psilocybin, with slightly less imagery and slightly more insightfulness. Strikingly, the response profile of racemic MDMA is very different from that of R(−)-MDMA, with no visual hallucinations and little to no insightfulness.

### Emotional Breakthrough Inventory (EBI)

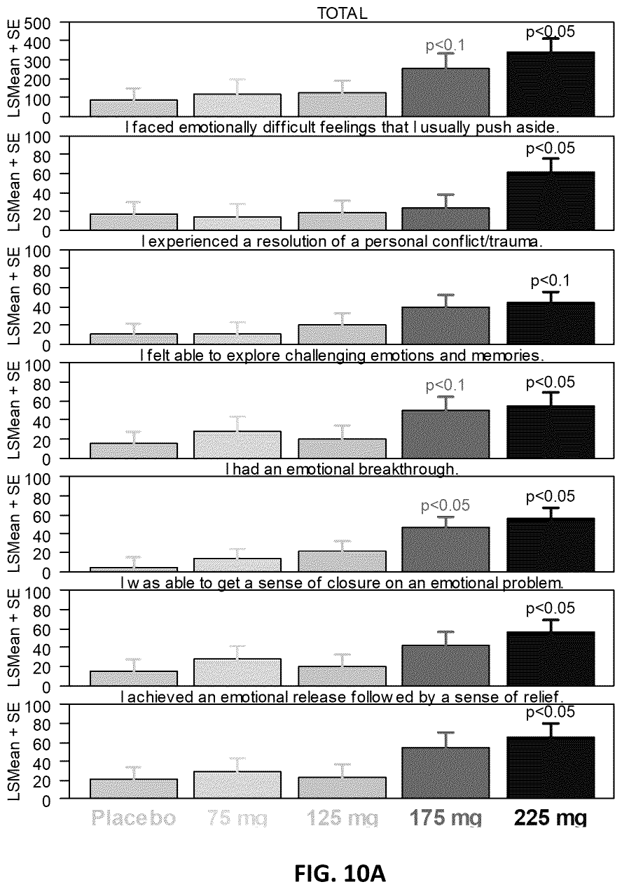

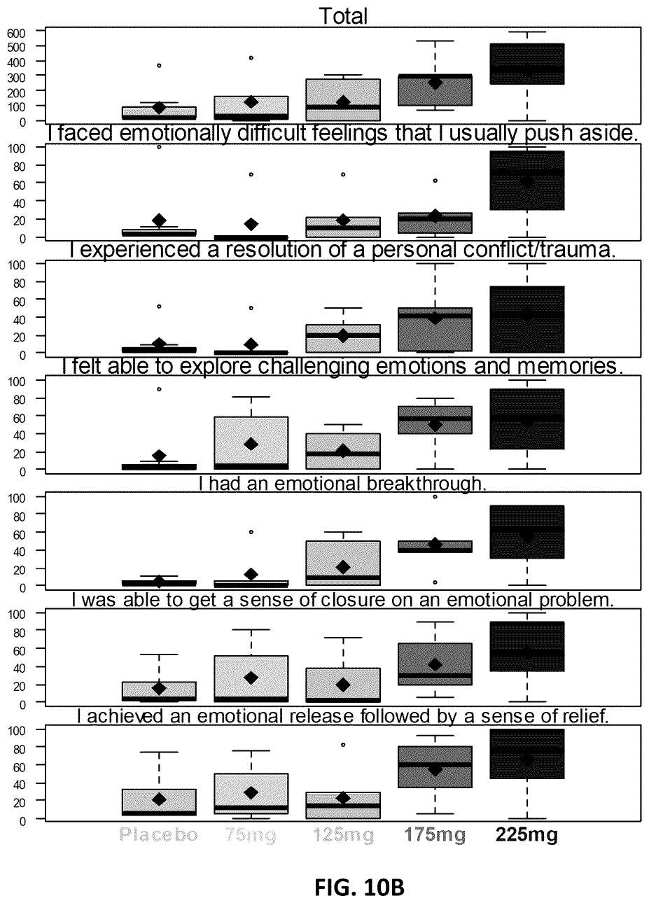

As illustrated in FIGS. 10A-10B, the Emotional Breakthrough Inventory (EBI) observations with R(−)-MDMA showed a clear dose-dependent response with statistical significance at higher doses. Self-reported breakthrough responses included: facing emotionally difficult feelings that are usually pushed aside; experiencing resolution of a personal conflict or trauma; ability to explore challenging emotions and memories; having an emotional breakthrough; achieving a sense of closure on an emotional problem; achieving emotional release followed by sense of relief.

### Self-Connectedness Scale

The Self-Compassion Scale (SCS) is a 12-item self-report measure that is used by adults to measure their capacity for self-compassion-the ability to hold one's feelings of suffering with a sense of warmth, connection and concern. Table 1 shows the changes from baseline as measured by SCS on Day 2 and Day 8 following R(−)-MDMA dosing. The results indicate that R(−)-MDMA induced a clear dose-dependent response with statistical significance at Day 8.

TABLE 1
Self-Connectedness Scale

| var | visit | _75_mg | _125_mg | _175_mg | _225_mg | Placebo |
|---|---|---|---|---|---|---|
| LSMean and 95% | Day 2 | 0.04(−0.32, | 0.23(−0.09, | 0.32(−0.04, | 0.16(−0.16, | −0.10(−0.41, |
| CI | | 0.40) | 0.55) | 0.68) | 0.48) | 0.20) |
| Difference with | Day 2 | 0.14(−0.36, | 0.33(−0.11, | 0.42(−0.04, | 0.26(−0.19, | |
| Placebo and 95% | | 0.63) | 0.77) | 0.88) | 0.71) | |
| CI | | | | | | |
| p-value | Day 2 | 0.5668 | 0.1299 | 0.0716 | 0.2450 | |
| LSMean and 95% | Day 8 | 0.08(−0.24, | 0.12(−0.17, | 0.12(−0.17, | 0.24(−0.05, | −0.28(−0.53, |
| CI | | 0.41) | 0.40) | 0.42) | 0.53) | −0.04 |
| Difference with | Day 8 | 0.37(−0.05, | 0.40(0.03, | 0.41(0.03, | 0.52(0.14, | |
| Placebo and 95% | | 0.78) | 0.78) | 0.79) | 0.91) | |
| CI | | | | | | |
| p-value | Day 8 | 0.0808 | 0.0367 | 0.0357 | 0.0088 | |

### Challenging Experiences Questionnaire (CEQ)

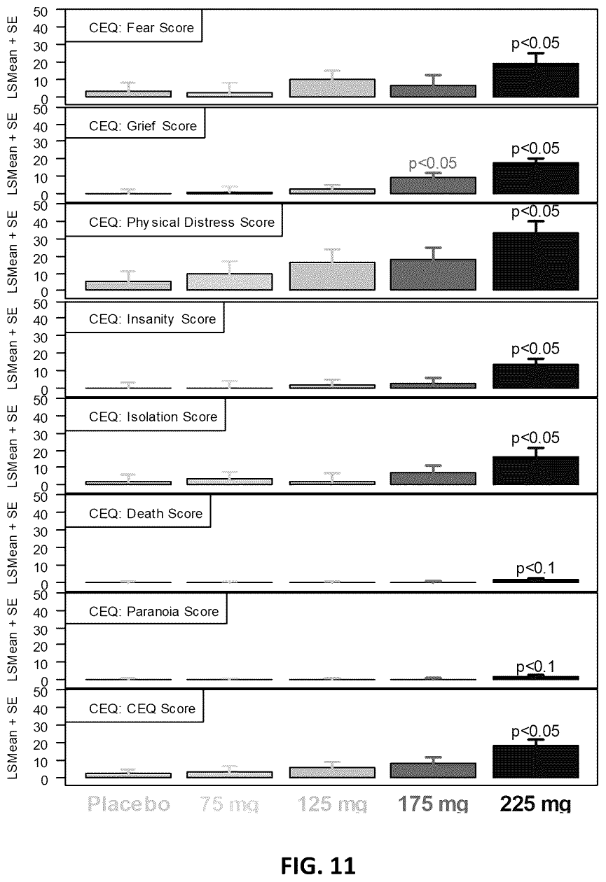

Seven Challenging Experiences Questionnaire (CEQ) factors (grief, fear, death, insanity, isolation, physical distress, and paranoia) provide a phenomenological profile of challenging aspects of experiences with R(−)-MDMA. As illustrated in FIG. 11, the changes from baseline at 6.5 hours post dosing showed a clear dose-dependent response with statistical significance at higher doses. These results also confirm the unexpected psychedelic-like response associated with R(−)-MDMA.

### Depression Anxiety Stress Scale

The Depression Anxiety Stress Scale (DASS) is a 42-item self-report instrument for measuring depression, anxiety and tension/stress. Table 2 shows the changes from baseline as measured by DASS on Day 8 following R(−)-MDMA dosing. The results show that R(−)-MDMA lowered anxiety at lower doses but increased anxiety at 225 mg compared to placebo.

TABLE 2
DASS results

| var | param | _75_mg | _125_mg | _175_mg | _225_mg | Placebo |
|---|---|---|---|---|---|---|
| LSMean and 95% | Depression | −1.03(−1.94, | −1.14(−1.96, | −0.91(−1.73, | 0.41(−0.58, | 0.88(1.62, |
| CI | | −0.12) | −0.32) | −0.08) | 1.41) | −0.14) |
| Difference with | Depression | −0.15(−1.26, | −0.26(−1.42, | −0.03(−1.08, | 1.29(−0.08, | |
| Placebo and 95% | | 0.96) | 0.90) | 1.02) | 2.66) | |
| CI | | | | | | |
| p-value | Depression | 0.78189 | 0.65162 | 0.95592 | 0.06302 | |
| LSMean and 95% | Anxiety | −1.22(−1.98, | −1.24(−1.95, | −1.26(−1.97, | 0.76(0.04, | −0.18(0.79, |
| CI | | −0.47) | −0.54) | −0.56) | 1.48) | 0.43 |
| Difference with | Anxiety | −1.04(−2.01, | −1.07(−2.01, | −1.08(−2.00, | 0.94(−0.03, | |
| Placebo and 95% | | −0.08) | −0.12) | −0.17) | 1.91) | |
| CI | | | | | | |
| p-value | Anxiety | 0.03518 | 0.02927 | 0.02163 | 0.05681 | |
| LSMean and 95% | Stress | −2.57(−4.41, | −2.54(−4.19, | −0.52(−2.23, | 0.05(−1.89, | −1.63(3.08, |
| CI | | −0.72) | −0.89) | 1.19) | 1.98) | −0.18) |
| Difference with | Stress | −0.93(−3.24, | −0.91(−3.12, | 1.11(−1.08, | 1.68(−0.84, | |
| Placebo and 95% | | 1.38) | 1.30) | 3.30) | 4.20) | |
| CI | | | | | | |
| p-value | Stress | 0.41310 | 0.40476 | 0.30629 | 0.18155 | |

A number of unexpected results were identified in the data from the Phase 1 study. The observed psychedelic-like effects associated with R(−)-MDMA were surprising, given the lack of documentation of similar effects in prior clinical trials with racemic MDMA. These unexpected results suggest that R(−)-MDMA possesses potential therapeutic efficacy for indications in which MDMA has previously been considered to be contraindicated and/or ineffective. For example, R(−)-MDMA is therapeutically effective for conditions where increased sociability, connectedness, and rejection sensitivity would be contraindicated, such as borderline personality disorder and depression. The results also suggest that R(−)-MDMA may be therapeutically effective for conditions associated with a lack of insightfulness, such as Cluster C personality disorders (e.g., avoidant personality disorder, dependent personality disorder, and obsessive-compulsive poersonality disorder). Further, the increase in serotonergic effects observed following R(−)-MDMA administration indicates that it may have greater therapeutic efficacy in treating depression than previously understood as activating the serotonergic system is known to improve mood and reduce fear, aggression, and impulsivity. In addition, R(−)-MDMA's ability to promote insightfulness, induce a blissful state, and lower anxiety may offer therapeutic benefits for social anxiety disorder (SAD). Enhanced insightfulness could help individuals with SAD gain a deeper understanding of the root causes of their social anxiety. The self-reflection can lead to more effective coping strategies and long-term improvements in anxiety management. Further, the blissful state induced by R(−)-MDMA can provide relief from the intense anxiety and fear associated with social situations, creating a more positive emotional baseline. Experiencing positive emotions can help patients reduce overal anxiety levels, making it easier to confront social fears.

### Example 2. Evaluation of the Effects of an Inhibitor of CYP2D6 on the Exposure of R(−)-MDA in Plasma or Brain of a Subject

A study will be conducted to evaluate the effects of an inhibitor of CYP2D6 on the exposure of R(−)-MDA in plasma or brain of a subject. A therapeutically effective amount of a CYP2D6 inhibitor to inhibit CYP2D6 metabolism of MDMA and a therapeutically effective amount of enantiomerically enriched MDMA are administered to a subject simultaneously or separately. The plasma or brain level of R(−)-MDA is measured at a series time points (e.g., 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 36, 48, 60, 72, 84 or 96 hours post administration).

### Example 3. Evaluation of the Effects of S(+)-MDMA on the Exposure of R(−)-MDA in Plasma or Brain of a Subject

A study will be conducted to evaluate the effects of S(+)-MDMA on the exposure of R(−)-MDA in plasma or brain of a subject. A therapeutically effective amount of a first composition comprising racemic MDMA or MDMA comprising S(+)-MDMA in an enantiomeric excess relative to R(−)-MDMA and a therapeutically effective amount of a second composition comprising enantiomerically enriched MDMA comprising R(−)-MDMA in an enantiomeric excess relative to S(+)-MDMA are administered to a subject concurrently or separately. The plasma or brain level of R(−)-MDA is measured at a series time points (e.g., 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 36, 48, 60, 72, 84 or 96 hours post administration).

### Example 4. Evaluation of R(−)-MDMA as a Treatment for a Social Anxiety Disorder

A subject who has been diagnosed with a social anxiety disorder will be administered with a therapeutically effective amount of a composition or a kit described herein. The subject will receive the following measures before and after the treatment: Liebowitz Social Anxiety Scale (LSAS), CGI-Severity of Illness scale, Social Phobia Inventory (SPIN) total score, social Connectedness Scale, Internalized Shame Scale, Quick Inventory of Depressive Symptomatology (QIDS), Columbia Suicide Severity Rating Scale, Self-Compassion Scale (SCS) total score, Psychotomimetic States Inventory (PSI), Challenging Experience Questionnaire (CEQ) total score, Emotional Breakthrough Inventory (EBI) total score, Mystical Experience Questionnaire, EQL-5D, Hamilton Rating Scale for Anxiety (HAM-A), Hamilton Rating Scale for Depression (HAM-D), axis V Social and Occupational Functioning Assessment Scale of DSM-IV, axis II (ICD-10) World Health Organization Disability Assessment, Schedule 2 (DAS-2), Sheehan Disability Scales, Schneier Disability Profile, World Health Organization Quality of Life-100 (WHOQOL-100), Quality of Life Inventory (QOLI), European Quality of Life 5-Dimensions scale, or other tests as described in Bobes, J. (1998).

1. A pharmaceutical composition comprising enantiomerically enriched MDMA comprising R(−)-MDMA in an enantiomeric excess relative to S(+)-MDMA, or a pharmaceutically acceptable salt thereof, an inhibitor of CYP2D6, and a pharmaceutically acceptable carrier, wherein the enantiomeric excess R(−)-MDMA is from about 50.01% to about 100%.

2. The pharmaceutical composition of claim 1, wherein the enantiomeric excess of R(−)-MDMA is about 65% to about 100%.

3. The pharmaceutical composition of claim 1, wherein the ratio of the inhibitor of CYP2D6 to enantiomerically enriched MDMA in the composition is from about 1:1 to about 1:500.

4. The pharmaceutical composition of claim 1, wherein the ratio of the inhibitor of CYP2D6 to enantiomerically enriched MDMA in the composition is from about 1:1 to about 1:25.

5. The pharmaceutical composition of claim 1, wherein the inhibitor of CYP2D6 comprises a reversible CYP2D6 inhibitor.

6. The pharmaceutical composition of claim 5, wherein the reversible inhibitor of CYP2D6 comprises quinidine, bupropion, terbinafine, perhexiline, abiraterone, cinacalcet, duloxetine, lorcaserin, mirabegron, amiodarone, celecoxib, clobazam, cobicistat, desvenlafaxine, diltiazem, diphenhydramine, Echinacea, febuxostat, fluvoxamine, gefitinib, hydralazine, hydroxychloroquine, imatinib, labetalol, methadone, pazopanib, propafenone, ranitidine, ritonavir, sertraline, telithromycin, verapamil, vemurafenib, terfenadine, progesterone, testosterone, lansoprazole, olanzapine, chlorpromazine, fluphenazine, haloperidol, thioridazine, risperidone, clozapine, trifluperidol, Panax ginseng, or Ginko biloba.

7. The pharmaceutical composition of claim 1, wherein the inhibitor of CYP2D6 comprises an irreversible CYP2D6 inhibitor.

8. The pharmaceutical composition of claim 7, wherein the irreversible inhibitor of CYP2D6 comprises cimetidine, pimozide, methamphetamine, metoclopramide, paroxetine, or desethylamiodarone.

9. A kit comprising enantiomerically enriched MDMA comprising R(−)-MDMA in an enantiomeric excess relative to S(+)-MDMA, or a pharmaceutically acceptable salt thereof, and an inhibitor of CYP2D6, wherein the enantiomeric excess of R(−)-MDMA is from about 50.01% to about 100%.

10. The kit of claim 9, wherein the enantiomeric excess of R(−)-MDMA is S(+)-MDMA is from about 65% to about 100%.

11. The kit of any of claim 9, wherein the ratio of the inhibitor of CYP2D6 to enantiomerically enriched MDMA is from about 1:1 to about 1:500, or from about 1:1 to about 1:25.

12. (canceled)

13. The kit of any of claim 9, wherein the inhibitor of CYP2D6 comprises a reversible CYP2D6 inhibitor.

14. The kit of claim 13, wherein the reversible inhibitor of CYP2D6 comprises quinidine, bupropion, terbinafine, perhexiline, abiraterone, cinacalcet, duloxetine, lorcaserin, mirabegron, amiodarone, celecoxib, clobazam, cobicistat, desvenlafaxine, diltiazem, diphenhydramine, Echinacea, febuxostat, fluvoxamine, gefitinib, hydralazine, hydroxychloroquine, imatinib, labetalol, methadone, pazopanib, propafenone, ranitidine, ritonavir, sertraline, telithromycin, verapamil, vemurafenib, terfenadine, progesterone, testosterone, lansoprazole, olanzapine, chlorpromazine, fluphenazine, haloperidol, thioridazine, risperidone, clozapine, trifluperidol, Panax ginseng, or Ginko biloba.

15. The kit of any of claim 9, wherein the inhibitor of CYP2D6 comprises an irreversible CYP2D6 inhibitor.

16. The kit of claim 15, wherein the irreversible inhibitor of CYP2D6 comprises cimetidine, pimozide, methamphetamine, metoclopramide, paroxetine, or desethylamiodarone.

17. A kit comprising (i) a first composition comprising racemic MDMA or MDMA comprising S(+)-MDMA in an enantiomeric excess relative to R(−)-MDMA, or a pharmaceutically acceptable salt thereof, and a pharmaceutically acceptable carrier; and (ii) a second composition comprising enantiomerically enriched MDMA comprising R(−)-MDMA in an enantiomeric excess relative to S(+)-MDMA, or a pharmaceutically acceptable salt thereof, and a pharmaceutically acceptable carrier.

18. The kit of claim 17, wherein the first composition comprises racemic MDMA.

19. The kit of claim 17, wherein the first composition comprises S(+)-MDMA in an enantiomeric excess relative to R(−)-MDMA.

20. The kit of claim 19, wherein the first composition comprises S(+)-MDMA in an enantiomeric excess of about 50.01% to about 100%, or about 65% to about 100%, or in an enantiomerically pure form.

21. (canceled)

22. (canceled)

23. The kit of any of claim 17, wherein the second composition comprises R(−)-MDMA in an enantiomeric excess of about 50.01% to about 100%, about 65% to about 100%, or about 90% to about 100%, or in an enantiomerically pure form.

24.-26 (canceled)

27. The kit of claim 17, wherein the combined amount of S(+)-MDMA and R(−)-MDMA in the first composition is effective to partially inhibit CYP2D6 metabolism of MDMA.

28. The kit of claim 17, wherein the weight ratio of the first composition to the second composition is from about 1:1 to about 1:1000, or from about 1:1 to about 1:25.

29. (canceled)

30. A method for sustaining responses to R(−)-MDA exposure in a subject comprising administering to the subject a therapeutically effective amount of the kit of claim 9.

31. The method of claim 30, wherein the subject has a CYP2D6 metabolic phenotype identified as ultra-rapid, extensive or efficient, or intermediate phenotype.

32. A method of increasing or prolonging exposure of R(−)-MDA in plasma or brain of a subject in need thereof, the method comprising administering to the subject a therapeutically effective amount of the kit of claim 9, wherein the therapeutically effective amount of the CYP2D6 inhibitor is in an amount effective to inhibit CYP2D6 metabolism of MDMA.

33. A method of increasing or prolonging exposure of R(−)-MDA in plasma or brain of a subject in need thereof, the method comprising administering to the subject a therapeutically effective amount of the kit of claim 17.

34. (canceled)

35. The method of any one of claim 32, wherein the CYP2D6 inhibitor is administered before the R(−)-MDMA, or at the same time as the R(−)-MDMA.

36.-51 (canceled)

52. The method of claim 33, wherein the weight ratio of the first composition to the second composition is from about 1:1 to about 1:1000, or from about 1:1 to about 1:25.

53. (canceled)

54. A method for treating social anxiety disorder in a subject in need thereof, comprising administering to the subject a therapeutically effective amount of the kit of claim 9.

55. The method of claim 54, wherein the effective amount of the inhibitor of CYP2D6 comprises a dose ranging from about 0.1 mg to about 750 mg.

56. The method of claim 54, wherein the effective amount of the enantiomerically enriched MDMA comprises a dose ranging from 1 mg to about 750 mg MDMA, or an equivalent dose of the pharmaceutically acceptable salt thereof.

57. The method of claim 54, wherein the CYP2D6 inhibitor and the enantiomerically enriched MDMA are administered concurrently, separately, or a combination thereof.

58. The method of claim 54, wherein the CYP2D6 inhibitor is administered within about 12 hours of administration of the enantiomerically enriched MDMA.

59. A method for treating social anxiety disorder in a subject in need thereof, comprising administering to the subject a therapeutically effective amount of the kit of claim 17.

60. The method of claim 59, wherein the effective amount of the first composition comprises a dose ranging from about 0.1 mg to about 750 mg, or an equivalent dose of the pharmaceutically acceptable salt thereof.

61. The method of claim 59, wherein the effective amount of the second composition comprises a dose ranging from about 0.1 mg to about 750 mg, or an equivalent dose of the pharmaceutically acceptable salt thereof.

62. The method of claim 59, wherein the first composition and the second composition are administered concurrently, separately, or a combination thereof.

63. The method of claim 62, wherein the first composition is administered within about 12 hours of administration of the second composition.
